(19) United States
(12) Patent Application Publication
(10) Pub. No.: US 2025/0171727 A1
(43) Pub. Date: May 29, 2025

(54) WIRELESS SENSORS FOR MONITORING AND CONTROLLING CIRCULATING BIOREACTOR

(71) Applicant: Capra Biosciences, Inc, Alexandria, VA (US)

(72) Inventors: Mark Poole, Raleigh, NC (US); Bonnie Harper, Aldie, VA (US); Matthew Harper, Aldie, VA (US); Elizabeth Onderko, Alexandria, VA (US); Andrew P. Magyar, Acton, MA (US)

(21) Appl. No.: 19/039,251

(22) Filed: Jan. 28, 2025

Related U.S. Application Data
(63) Continuation of application No. PCT/US2023/071992, filed on Aug. 10, 2023.
(60) Provisional application No. 63/396,634, filed on Aug. 10, 2022.

(51) Int. Cl. C12M 1/34; C12M 1/00; C12M 1/21; C12M 1/36

(52) U.S. Cl. CPC C12M 41/34; C12M 41/02; C12M 41/06; C12M 41/28; C12M 41/32; C12M 41/44; C12M 41/48

(57) ABSTRACT

A system for producing a hydrophobic chemical includes circulating biofilm bioreactors for synthesizing the hydrophobic products and continuous extraction into an organic solvent phase. Various configurations can be employed for assembling the bioreactors into a production module. Extraction operations can be conducted in one or more extraction vessels. In some cases, product and solvent are separated using membrane separation techniques, with solvent being rerouted to the extraction vessel(s). Methods for the biotransformation of feedstocks into chemical products also are described.

### RELATED APPLICATIONS

This application is a continuation of International Application No. PCT/US2023/071992, filed on Aug. 10, 2023, now International Publication No. WO 2024/036256, published on Feb. 15, 2024, which International Application claims the benefit under 35 USC 119 (e) of U.S. Provisional Application No. 63/396,634, filed on Aug. 10, 2022, both of which are incorporated herein by reference in their entirety.

This application is related to U.S. Application number ______, filed on an even date herewith, entitled “CIRCULATING BIOFILM BIOREACTOR”, attorney docket number 0412.0004US1, now U.S. Patent Publication No.: _______; and U.S. Application number ______, filed on an even date herewith, entitled “CIRCULATING BIOREACTOR WITH BUBBLE TRAP”, attorney docket number 0412.0004US2, now U.S. Patent Publication No.: ______, both of which are incorporated herein by reference in their entirety.

### REFERENCE TO AN ELECTRONIC SEQUENCE LISTING

An electronic Sequence Listing conforming to the rules of WIPO Standard ST.26 is hereby incorporated by reference in its entirety. The contents of the electronic Sequence Listing are encoded as XML in UTF-8 text. The electronic document, created on Jul. 18, 2023, is entitled “0412.0004 WO1_ST26.xml”, and is 92,039 bytes in size.

### BACKGROUND OF THE INVENTION

In industrial scale biomanufacturing, bioreactors or fermenters are used to grow cells to produce chemical products, typically in suspended cell reactors during so-called log-scale growth where biomass is undergoing periodic doublings. To produce hydrophobic products via these traditional fermentation methods, one typically grows the biomass, harvests the cells, and then extracts the products from the cells.

Recently, a biofilm bioreactor, described in US Application Pub. No. US 2021/0253990, which is incorporated herein by this reference in its entirety, was developed. This biofilm bioreactor allows continuous product synthesis and extraction into an immiscible solvent phase. It uses naturally forming biofilms of hydrocarbonoclastic and/or oleaginous organisms growing on a solid support in a packed bed reactor.

### SUMMARY OF THE INVENTION

Key challenges for achieving high product yield from aerobic micro-organisms in a packed bed biofilm reactor are oxygenation, achieving substantial biomass, the routing and separation of the immiscible fluids.

The present invention relates to an apparatus, system and method for producing chemicals using a biofilm reactor in which supported or self-aggregated biofilm microorganisms are suspended in the reactor medium. While in previous techniques the biofilm and support material remain in a fixed location and solvent is circulated over the biofilm, the present approach features suspended solid supports carrying the biofilm (or suspended biofilm self-aggregates) that are pumped from one type of reservoir (the synthesis vessel) into a second type of reservoir (the extraction vessel).

In general, the microorganism is selected for its intrinsic characteristics that provide reaction pathways in which a carbon source is converted to a desired product. In many embodiments, the organism is tolerant to organic solvents, a property that enables two phase extraction of products while preserving functional biomass.

Aspects of the invention feature a system that includes one or more synthesis (growth) vessels and one or more extraction vessels.

In the synthesis vessel, biofilm on a solid support (beads, for instance) or self-aggregated biofilm, is fluidized by air and/or liquid, e.g., water pressure. Cells in the biofilm convert a carbon source into a product such as, for instance, a hydrophobic chemical. The carbon source is a compound or a mixture of compounds that can be converted by a hydrocarbonoclastic and/or oleaginous organism into the desired (hydrophobic) chemical. In examples, the carbon source is introduced into the synthesis vessel as a feedstock or as a component thereof. Many embodiments employ an aqueous feedstock.

In the extraction vessel, solid supports carrying the biofilm, biofilm self-aggregates and/or any planktonic cells present in the media extracted from the bioreactor are circulated through an organic solvent to extract accumulated product.

Multiple synthesis vessels can be combined to form a production module or assembly. In some embodiments, the vessels can be arranged in a parallel configuration for continuous operation while in other embodiments the vessels are arranged in a serial fashion such that feedstock and biomass is circulated from one column into the next sequentially until finally reaching the extraction vessel. In examples, aeration is provided to some or each column.

An assembly configuration in which the vessels are arranged in series also can be employed. In some implementations, aeration is provided to selected columns of the series.

A single synthesis vessel or a set comprising multiple synthesis vessels can be used in conjunction with a single extraction vessel or with a plurality of extraction vessels. In many implementations, the extraction vessel features layers in which media, typically water-containing, occupies the bottom region of the vessel, while a lighter solvent floats on top. An extraction module, including one or more extraction vessels, can further include a separator, which, in one example, utilizes a filtration membrane for isolating product from solvent. The solvent can be returned to the extraction vessel, while the product can be collected or further processed.

Some of the features or and possibly characterizing aspects of the invention include one and often more than one of the following attributes. For some embodiments, for instance, an oxygen-containing gas, often sterile air, is introduced into each column (or into selected columns in a production module) and diffused to ensure adequate dissolved oxygen. A flow controller to control the introduction of air can be provided in all or in selected vessels. A feed source for the biofilm microorganisms is introduced as needed and the introduction can be monitored. In some implementations, acid and/or base can be added, to maintain a pH-controlled environment in the synthesis vessels. Waste can be removed continually or periodically to ensure a healthy bioreactor operation. Sensor(s) for monitoring the pH, dissolved oxygen (DO), temperature, pressure, carbon composition, etc. can be installed in one or more synthesis column(s) and/or in one or more extraction vessel(s). The sensors can be hard-wired or wireless. In some embodiments one or more wireless sensors circulate through the system or components thereof (specific vessels, specific modules, etc.).

In some implementations, the extraction vessel is configured for the simultaneous separation of solvent, media and air. A condenser column can be included to recapture solvent. Some examples utilize a carbon filter to prevent solvent leakage. Air vent filters can be provided to allow the release of (sterile) air.

Specific embodiments feature a filtration loop attached to the extraction vessel, making possible the continuous removal of solvent and product.

In some of its aspects, the invention features a bioreactor design having two or more sets of vessels. One implementation includes a set of vessels in which biofilm on a solid support is fluidized by air and/or water pressure and cells in the biofilm convert a carbon source into a product. In the second set of vessels, the solid supports carrying the biofilm along with any planktonic cells in the media are circulated through an organic solvent to extract accumulated product.

Thus, in a first embodiment, a bioreactor includes two vessels, the first, the synthesis vessel, comprising a solid support suitable for the growth of biofilm and providing containment for a feedstock solution (typically an aqueous solution containing a carbon source, minerals, vitamins, and/or other nutrients) in contact with a biofilm. The second, the extraction vessel, comprises a container of extraction solvent. One or more pumps can be used to keep the solid support fluidized, freely suspended in the feedstock solution. The pump(s) can also serve to transfer the suspended bead mixture from the first vessel into the second vessel (containing the extraction mixture), and back to the first vessel.

In a second embodiment, the bioreactor has multiple synthesis vessels comprising a solid support suitable for the growth of biofilm and providing containment for a feedstock solution (e.g., an aqueous solution containing a carbon source, minerals, vitamins, and/or other nutrients) in contact with a biofilm. The multiple synthesis vessels interface with a single extraction vessel or with multiple extraction vessels that contain(s) the extraction solvent. An extraction module, containing one or more extraction vessels, can further include associated equipment for the separation of phases. One or more pumps can be used to keep the solid support fluidized, freely suspended in the feedstock solution. The pump(s) can also serve to transfer the suspended bead mixture from the synthesis vessels into the extraction vessel.

In some embodiments, the solid support is omitted, and the microbes are allowed to form aggregate biofilms for the synthesis of products. These aggregate biofilms become the “solid support” over time and remain suspended in solution during the process.

As used herein, the term “support” will encompass biofilm aggregates (that, as the process progresses, will support added microorganisms), and also the conventional solid supports (beads, for instance) that are made from a material (e.g., plastic, glass, etc.) that is different from the biofilm. In the case of aggregates, the “support” can include dead or live microorganisms. As used herein the terms “biofilm particles” or “suspended biofilm particles” will generally refer to biofilm on solid supports (beads, for example) as well as to self-aggregated biofilm, the latter not being formed on a support other than one formed from the microorganisms themselves.

Generally, whether supported or in the form of self-aggregates, the suspended biofilm particles are encouraged to remain suspended and generally not allowed to settle outside a designated settling tank in many examples.

In some embodiments, an insert is placed at the base of the synthesis vessel to induce vortices and/or angular flow. The angular flow can increase mixing, minimize jamming of the solid support particles, and reduce required pumping force.

In other embodiments, gas is pumped into one or more reservoirs. This gas can provide additional buoyant force for the biofilm material and introduce oxygen into the synthesis vessel(s). In further embodiments, an oxygen-containing gas is supplied to the synthesis vessel(s) and an inert gas is added to the extraction vessel to create an anoxic environment.

In yet another embodiment, the production module includes reservoirs, pumps, and valves to allow for the introduction of additional carbon-containing feedstock or various required nutrients. A sensor unit that can be attached to the bioreactor assesses the bioreactor conditions, and communicates with a central processor, which then controls pumps and valves to introduce nutrients into the reactor.

In a further embodiment, a membrane in a separation (e.g., filtration) loop interacts with the extraction vessel, serving to concentrate product and return pristine solvent to the extraction vessel. In certain implementations, a pump is used to remove the solvent-based feed from the extraction vessel and create a pressure across the membrane.

The invention also relates to a method for the synthesis of hydrophobic products using an hydrocarbonoclastic and/or oleaginous organism.

Thus, as a result of the invention and its embodiments, an improved apparatus and method that provides sufficient aeration with simplified extraction is now available for the biomanufacturing of hydrophobic chemicals.

In general, according to one aspect, the invention features a circulating bioreactor system for producing chemical products. The circulating bioreactor system comprises one or more synthesis vessels, an extraction vessel, a controller, and wireless sensors. The one or more synthesis vessels are configured to support the growth of microorganisms and facilitate the conversion of a carbon source into a desired product. The extraction vessel is configured to extract the desired product from the media into an organic solvent. The wireless sensors, which are operably deployed within the bioreactor system to monitor process parameters, transmit data of the process parameters to the controller.

In embodiments, the wireless sensors include pH, dissolved oxygen, carbon dioxide concentration, temperature, pressure, flow rate, and/or feedstock composition, to name a few examples. The wireless sensors are configured to measure biomass density and biofilm health within the synthesis vessels and/or monitor solvent levels and product concentration in the extraction vessel, in other examples. The wireless sensors monitor air in the one or more synthesis vessels, waste of the bioreactor system, product of the bioreactor system, a feed from a feed reservoir of the bioreactor system, and/or monitor a solvent layer and/or a media reservoir layer within the extraction vessel, for example.

The wireless sensors may be distributed throughout the bioreactor system, monitoring conditions in multiple locations, including the synthesis vessels, extraction vessel, and/or connecting conduits. In some examples, the wireless sensors are configured to circulate within the bioreactor system along with the media flow to provide spatially distributed measurements. In some embodiments, the wireless sensors include sensors that are embedded in a reactor base or wall of the bioreactor system.

The wireless sensors include optical sensors configured to measure process parameters through fluorescence or absorbance spectroscopy and/or electrochemical sensors configured to perform cyclic voltammetry or electrochemical impedance spectroscopy for monitoring process parameters.

In one example, the wireless sensors are configured to transmit data in real time to the controller, enabling dynamic adjustments to process parameters.

The controller uses data from the wireless sensors to adjust gas flow rates, nutrient feed rates, or circulation flow rates within the bioreactor system.

In other examples, the wireless sensors are configured to detect deviations from normal process parameters for the controller, monitor redox conditions and oxygen levels to enable the creation of oxidative and reducing zones within the bioreactor system, detect foam levels in the extraction vessel and transmit data to the controller for foam control (e.g., via height sensors of the wireless sensors), measure optical density to monitor cell growth and adjust nutrient introduction accordingly, and/or monitor and optimize phase separation between the media and organic solvent in the extraction vessel.

In some embodiments, the wireless sensors include sensor arrays.

In another example, the controller activates or adjusts a flow controller, allowing more or less gas to the one or more synthesis vessels, based on the data of the process parameters received from the wireless sensors.

The wireless sensors include a level height sensor (e.g., an optical line break sensor) for detecting foam generation in the extraction vessel.

In general, according to another aspect, the invention features a method of operating a circulating bioreactor system for producing chemical products. The bioreactor system comprises one or more synthesis vessels configured to support the growth of microorganisms and facilitate the conversion of a carbon source into a desired product, an extraction vessel configured to extract the desired product from the media into an organic solvent, and a controller. The method comprises wireless sensors that are operably deployed within the bioreactor system monitoring process parameters and transmitting data of the process parameters to the controller.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention.

The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1. is a schematic diagram of a system that includes multiple bioreactors in a parallel configuration, an extraction vessel and a separator according to the invention;

FIG. 2 is a longitudinal cross-section of an exemplary bioreactor showing its dimensions in centimeters (cm);

FIGS. 3A and 3B are schematic diagrams of systems that include multiple bioreactors connected in series, an extraction vessel and a separator according to the invention;

FIG. 4 is a schematic diagram of an extraction vessel, the blow-up section illustrating components present in an intermediate layer during operation;

FIG. 5 is a schematic diagram of another embodiment of a system that includes multiple bioreactors, a bubble trap and a two-phase extraction vessel according to the invention;

FIG. 6 is a diagram of the two-phase extraction vessel of FIG. 5 according to the invention;

FIG. 7 is a diagram of the two-phase extraction vessel of FIG. 5, further including a filtration loop attached to the solvent circulation loop according to the invention;

FIG. 8 is a block diagram depicting how various process parameters are controlled based on sensor information in the controller 200; and

FIGS. 9A through 9D show several designs of a mixer that can be employed in practicing aspects of the invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise.

It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The apparatus, system and method described herein preferably employ hydrocarbonoclastic and/or oleaginous microorganisms to synthesize a desired product, a hydrophobic chemical, for instance, that can then be separated out or extracted. Synthetic approaches rely on suspending microorganism particles in a vessel or reactor.

In many of its aspects, the invention uses a solid phase (or matrix) in the form of a particulate material, for example, to support a biofilm of hydrocarbonoclastic and/or oleaginous microorganisms. The solid phase can be provided in the form of granules or beads, such as silica or glass beads, for example. Other possible supports include but are not limited to magnetic beads, wood chips, plastic beads, etc.

Surprisingly and unexpectedly, it was discovered that processes described herein can be carried out even if the support is omitted. It was found, for instance, that dispensing with the support, namely the solid phase in particulate form, beads, for instance, did not hinder and could, in fact, enhanced efficiency. In the absence of a support, the microorganisms self-assemble, forming aggregates, referred to herein as “self-aggregates”. Thus, accordingly, some aspects of the invention use biofilm microorganisms that self-aggregate, forming biofilm particles, e.g., of 10 microns or more, for example. In illustrative examples, the self-aggregates can have a diameter or largest dimension within a range of from about 1 millimeter (mm) to about 3 centimeter (cm). Self-aggregation can occur with microorganisms such as Escherichia coli, Pseudomonas aeruginosa, Pseudomonas stutzeri, and Clostridium thermocellus, to list a few. Furthermore, the formation of self-aggregates can occur with live as well as dead biofilm microorganisms, where living, productive cells form a thin active biofilm on a core of dead cells. The dead cells may also can serve as a carbon source for the live cells.

As with their supported counterparts, the biofilm self-aggregates are maintained in a suspended state during the synthesis or production process. If desired, settling of supported or self-aggregated biofilm can take place in a designated settling tank.

Whether supported or unsupported, the growth or maintenance of biofilm microorganisms is sustained by a carbon source (often provided in an aqueous media feed). The carbon source is converted into chemical products by biosynthetic pathways contained within the organism. Examples of carbon sources that can be employed include but are not limited to: acetate, succinate, lactate, glycerol, ethanol plant byproducts, e.g., thin stillage, food waste byproducts, anaerobically digested food waste, agricultural waste byproducts, fermentation byproducts or others, as well as combinations thereof.

A biofilm-forming microbial community can be used as a microbial catalyst, where different microbial species can metabolize different components of a thin stillage or fermentation byproduct or catalyze different steps of product formation.

The system described herein includes a synthesis vessel (also referred to herein as a “synthesis (or production) column”, “bioreactor” or “reactor”, and an extraction vessel (or column). In many implementations, the synthesis vessel receives fluids (liquid and/or gas) from the bottom of the column. Bioreactor content can be withdrawn from the top of the synthesis column and fed to the top of an extraction vessel, where product is removed from the cells.

The synthesis vessel or bioreactor is configured to create a high-density suspension of a granular material suitable for growth typically of oleaginous and/or hydrocarbonoclastic organisms such as Marinobacter spp., and allow for the continuous or periodic circulation of the biofilm suspension through an organic solvent. This design differs from previous biofilm bioreactor designs, such as that described in U.S. Application Pub. No. US 2021/0253990, where the biofilm and support material remain in a fixed location and solvent is circulated over the biofilm. In the present approach, the biofilm self-aggregates or the solid support carrying the biofilm is pumped from one reservoir (the synthesis vessel) into a second reservoir (the extraction vessel).

For many applications, the synthesis vessel is designed to provide a well-mixed, carbon-, nutrient-, and oxygen-rich environment organisms forming the biofilm to convert a carbon source (e.g., acetate, succinate, thin stillage components, etc.) provided in the aqueous feedstock.

In specific embodiments, the support material is kept in suspension by continuously pumping the aqueous media and support suspension (if supported biofilm is employed) from the extraction vessel into the bottom inlet of the synthesis vessel. If no supports, e.g., beads, are used, the stream being returned to the bioreactor will contain biofilm self-aggregates suspended in the aqueous media.

Air or other gases or gas mixtures also can be pumped into the synthesis vessel, typically from the bottom of the vessel. The gas can serve both as a supplemental source of lift for the biofilm-support (or biofilm self-aggregates), as well as a crucial source of oxygen for the intensive metabolic processes taking place in the biofilm microorganisms. In some cases, the gas is oxygen-enriched air, to further increase aeration in the reactor. The oxygen-containing gas, e.g., sterile air, can be introduced into a column and diffused, to ensure adequate dissolved oxygen (as monitored by sensors that are either circulating or fixed inside the reactor). In other cases, the gas, e.g., air, is enriched with CO₂, to help buffer the growth medium.

While a single synthesis vessel can be employed, many embodiments feature a plurality or multiple (i.e., two or more) vessels. Three, four and as many as 10,000, e.g., at least 200, 500, 800, 1000, 2000, 3000, 4000, 5000, 6000, 7000, 8000 or 9000, can be combined in a synthesis or production module or assembly. Configurations that can be employed include, for instance, columns arranged in parallel or in series.

The extraction vessel can be part of an extraction module or loop that can further include separation equipment for isolating product from the solvent. Further operations can be conducted to collect the product and/or recycle the solvent.

Once in the extraction vessel, the media, cells, biofilm, and, in some cases, solid supports, e.g., beads, that have been transferred from one or more bioreactors fall through the solvent phase due to gravity. As they transit through the solvent layer, hydrophobic products are extracted into the hydrophobic phase (solvent). The productivity/viability of the biomass is maintained through the extraction process due to the innate tolerance of the biofilm-forming organism to hydrophobic organic solvents.

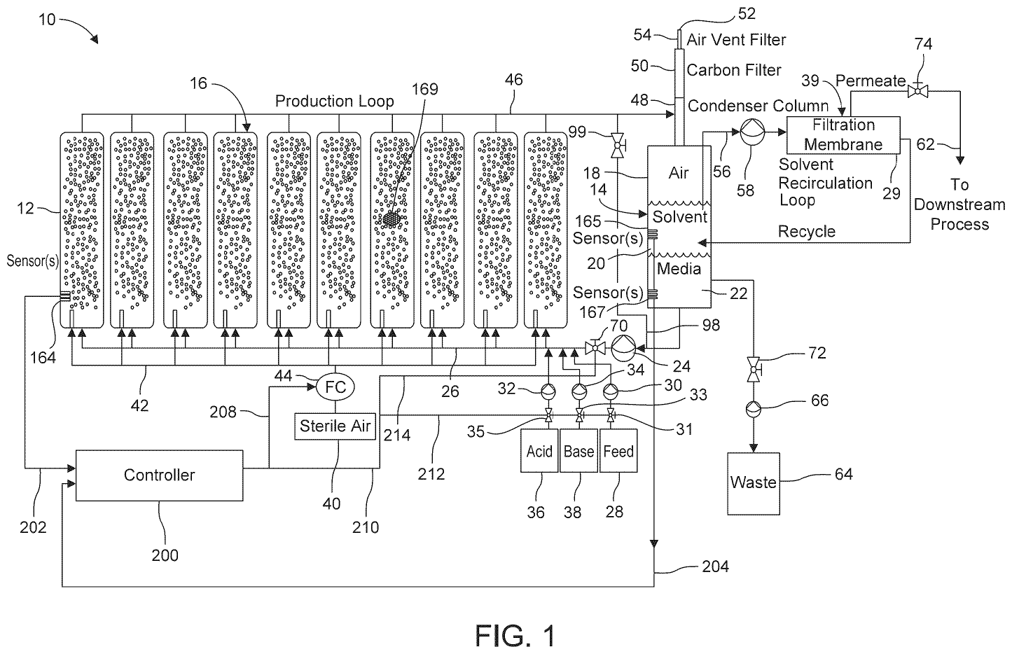

Shown in FIG. 1 is system 10 including ten synthesis vessels 12, arranged in a parallel configuration, and extraction vessel 14.

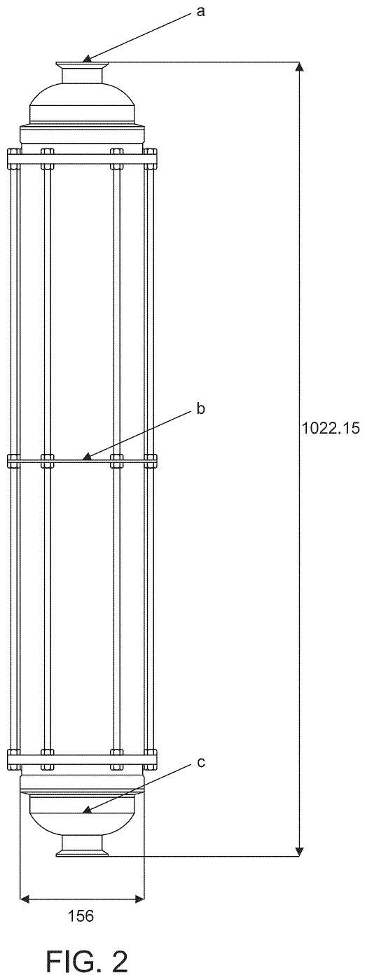

Synthesis vessel 12, also referred to herein as a “synthesis column” “bioreactor” or “reactor”, can be constructed from a suitable material such as a plastic material (e.g., acrylic), glass, metal (e.g., aluminum, steel), fiberglass, carbon fiber, and so forth and can have a capacity within a range of from about 1 liter (L) to about 100,000 L. In many cases, the synthesis vessel has a cylindrical shape, but other geometries can be employed. Squat or short columns as well as tall columns can be selected, depending on process parameters, the desired residence time, available footprint, ceiling heights, or other factors. In one example, the synthesis vessel 12 is cylindrical and has a height within a range of from about 3 feet (ft) to about 20 ft, and a diameter within a range of from about 0.25 ft to about 3 ft. A longitudinal cross-sectional view of an illustrative synthesis vessel 12 with dimensions in centimeters (cm) is presented in FIG. 2. Specifically, the vessel in this figure has a diameter of 156 cm and a height of 1022.15 cm.

In a typical approach, a synthesis (also referred to herein as “production”) module or “loop” (e.g., module 16 in FIG. 1) will include the same type of columns 12. Different column designs can be employed in some cases.

The extraction vessel 14 can be fabricated from a suitable material, e.g., a material similar to that employed in constructing the synthesis vessel. For continuous operations, it can have a capacity that depends on the capacity of the synthesis module or on other parameters such as flow rate, productivity (e.g. grams product/L/hr), product stability, and others. A suitable geometry, cylindrical for instance, can be selected, with a height within a range of from about 1 ft to about 20 ft, and a diameter within a range of from about 1 inch (in or ″) to about 2 ft.

Extraction vessel 14 can feature a gas (e.g., air) gap 18, a solvent layer 20, and a media reservoir 22. In specific embodiments, the solvent layer 20 is formed of a hydrophobic extraction solvent, such as hexane, heptane, dodecane, oleic acid, soybean oil, castor oil, corn oil, avocado oil, another suitable water immiscible solvent or combinations thereof. Beneath the solvent layer, media reservoir layer 22 typically contains an aqueous composition from which the solvent spontaneously phase separates. Because of the lower density of the solvent phase, the solvent layer remains on top of the aqueous phase.

The system can also include a separation arrangement or loop 39 that uses filtration through membrane system 29, for instance. Specific examples use a chemically-selective or a size-selective membrane, gravity or centrifugal separation, or a column based separation similar to chromatographic separations. The separation loop or separation module can be interfaced with the solvent layer 20, to remove, e.g., continuously, product from the system and recycle the solvent back to the extraction vessel.

Certain product molecules, retinoids, for instance, are highly sensitive to oxygen. The environment inside the cell is reducing, so prior to their extraction, these molecules are maintained in their desired state. Once extracted from the cells, however, these substances can begin to degrade. To reduce oxidation, the extraction vessel can be purged with an inert gas such as nitrogen, thus reducing or preventing oxygen exposure of the product, as the molecules are extracted into the solvent phase.

Media exits the media reservoir layer 22 at the bottom of extraction vessel 14 and is fed, using pump 24, to manifold 26 which connects to the bottom of each reactor 12.

Optionally, supplemental fresh media can be added from a reservoir, e.g., reservoir 28, using pump 30. Materials such as an acid or a base, pumped, respectively by pumps 32 and 34, can be added as needed from reservoirs 36 and 38 under the control of controller 200. Flow from the reservoirs can be controlled by motorized ball valves 31, 33 and 35 operated by the controller 200.

In some embodiments, air, a nitrogen, oxygen mixture, or another suitable gas is added to a reservoir, e.g., reservoir 28, providing a desired environment for the feed media and adding to the buoyancy for the supported or self-aggregated biofilm. Certain gases or gas mixtures, e.g., CO, CO2, Methane, Ethane, etc, may also serve as a carbon source for the organism.

Gas is supplied to each column 12 from gas source 40, e.g., a sterile air source, in the direction of the arrows. Oxygen gas or oxygen-enriched air can be used in addition or as an alternative to air.

In the arrangement of FIG. 1, the gas is fed through manifold 42. Other approaches can be implemented. For example, the gas can be fed through the same manifold employed for the media feed. Flow rates can be monitored or controlled by device 44, e.g., a mass flow controller under the control of the controller 200.

Generally, gases can be introduced in any one of the columns or vessels described herein using a device such as, for example, a diffuser stone, sparger, drilled (or perforated) piping or other equipment that can produce gas bubbles, in particular small (fine) gas bubbles that enhance dissolution of the gas into the liquid medium. In many implementations, the gas is fed from the bottom of the synthesis vessel and a bubble-producing device, e.g., a diffuser stone is positioned at the bottom of the vessel. Other arrangements can be considered, however.

In some embodiments, different gas compositions or flow rates can be applied in different columns. This can generate different synthetic zones, where the environment can be more oxidative or more reducing.

As an alternatively or in addition to the bubble generating device (e.g., a diffuser stone), the synthesis vessel can be provided with an oxygen permeable tubing made, e.g., from a fluoropolymer (e.g., fluoroethylenepropylene), low density polyethylene, silicone. The tubing can increase gas (e.g., air) transport in the bioreactor without generating gas bubbles and, in specific examples, receives air or another suitable gas from an inlet port at the bottom of the synthesis vessel, and extends through the interior of the vessel to an outlet port. In one implementation, the inlet and outlet ports are arranged in a staggered configuration relative to one another, resulting in a configuration in which the permeable tubing is not parallel to the vertical axis or the bioreactor. Other arrangements, e.g., spiral tubing, coiled tubing, multiple tubes connecting pairs of inlet and outlet ports, etc. also can be employed.

Contents from the reactors are withdrawn from the top of columns 12 via manifold 46 and are directed to the top or extraction vessel 14. Condensation column 48 can be installed at the top of vessel 14 to capture solvent vapors and return them to the extraction vessel. Gas, e.g., air, exiting from the condensation column can be passed through carbon filter 50 (which controls solvent leakage), then released at vent 52, which can be provided with air vent filter 54, to ensure release of sterile air.

Product-containing solvent from solvent layer 20 is directed (via conduit 56) to separation arrangement 39, which can include one or more chemically-selective and/or size-selective membranes. The membrane separates the feed stream (product-containing solvent) into its components. In one embodiment, solvent passes through the membrane material (as the permeate fraction), while the product, e.g., a hydrophobic chemical, is retained (as the retentate or concentrate fraction).

Another embodiment includes two membranes. In the first, stream is concentrated (purified solvent passing through as permeate), while in the second membrane product is removed as permeate.

In yet another multi-membrane embodiment, a first membrane has a smaller size than the target molecule and a second membrane has a larger size than the target molecule. In the first stage, the permeate would be returned the reactor, and the retentate sent to the second stage. This will concentrate the product in the solvent. In the second state, the protein will pass through the membrane, eliminating any cellular debris or larger proteins, while the retentate would be returned to the reactor. The product can be collected for further purification such as affinity tag purification or other methods of chromatographic separation.

In another embodiment, the product and solvent go through the first membrane to remove impurities; in the second membrane, a second immiscible solvent is mixed in, and the product and second solvent is removed with the second membrane.

A high-pressure (e.g., 0 to 800 psi) pump 58 controlled by controller 200 can be added to generate the driving force needed to push the solvent through the membrane. This solvent is then routed back (via conduit 60) to the solvent layer 20 of the extraction vessel. The retained (isolated) product is collected as the membrane fraction that exits at outlet 62. From there, it can be subjected to further downstream operations, such as further purification, compounding, or other steps.

Waste 64 can be withdrawn from media layer 22 continuously or intermittently using pump 66.

Valves such as valves 70, 72, 74 or 76, etc. can be used to open, close, and/or adjust flow under the control of the controller 200. Conduit 98 and valve 99 can be used to bypass the extraction vessel and recycle media to the production loop to circulate the biofilm reactor without going through the extraction vessel.

One, selected columns or all the columns in production module 16 can be provided with sensors 164, which can form sensor arrays. In some embodiments, the sensors are hard-wired to the controller 200. In addition or in the alternative, one or more or all the columns can be provided with wireless sensors (e.g., sensor 169) that typically will communicate with a wireless interface board outside the bioreactor and typically part of the controller 200. These wireless sensors may include individual elements to measure pH, oxygen, fluorescence-based sensing, or take electrochemical measurements via cyclic voltammetry, electrochemical impedance spectroscopy or other techniques. The data produced by these sensors is received an analyzed by the controller 200.

Non-limiting examples of wireless sensors are described, for example, in, “A Threshold-Based Bioluminescence Detector With a CMOS-Integrated Photodiode Array in 65 nm for a Multi-Diagnostic Ingestible Capsule”, Liu, Q., Jimenez, M., Inda, M.E., Riaz, A., Zirtiloglu, T., Chandrakasan, A.P., Lu, T.K., Traverso, G., Nadeau, P. and Yazicigil, R.T., 2022. A Threshold-Based Bioluminescence Detector With a CMOS-Integrated Photodiode Array in 65 nm for a Multi-Diagnostic Ingestible Capsule. IEEE Journal of Solid-State Circuits, 58(3), pp.838-851. Other types of wireless sensors, as known in the art or as developed in the future, can be employed.

Sensors 164 can be placed at the bottom of each or selected columns 12, attached to an internal surface in a column, or at another suitable location. Sensors that circulate with the flow in the circulating reactor also can be employed. Some implementations utilize wireless sensors that actively circulate along the flow path with the cells through the reactor system. Sensors also can be placed in conduits to assess conditions and/or components of fluids passing through. The type of sensors used and/or how the sensors are deployed within a vessel can be the same or can differ from column to column.

Sensors, e.g., such as described above, also can be provided in an extraction vessel to enable monitoring by the controller 200. For example, in the embodiment of FIG. 1, vessel 14 is monitored by sensors (or sensor array) 165 in solvent layer 20 and from sensors (or sensor array) 167 in the media layer 22.

The controller 200 is configured to receive inputs from various sensors in one, more or all columns in production module 16, and/or inputs from sensors in at least one extraction vessel 14. In one example, controller 200 receives input 202 from sensors 164, inputs 204 from sensors 165 (in solvent layer 20) and from sensors 167 (in the media reservoir layer 22).

Based on data received (oxygen levels, pH, chemical content, etc.) from the various sensors, controller 200 will issue various responses, such as controlling the air feed (output signal 208) via flow control device 44; controlling valve 70 (output signal 210) for; and/or controlling one or more valves 31, 33 and 35 (output signal 212) to enable feedback control of the process.

Communications to and/or from controller 200 can be cabled (wired) or wireless.

It is possible to operate all columns in the same manner, transferring contents to the extraction module simultaneously. In many embodiments, however, each column (or a group of columns) within production module 16 is operated independently from the remaining columns in the module. Biofilm-containing media will be transferred from some columns but not from others, depending on the stage reached in the synthesis vessel.

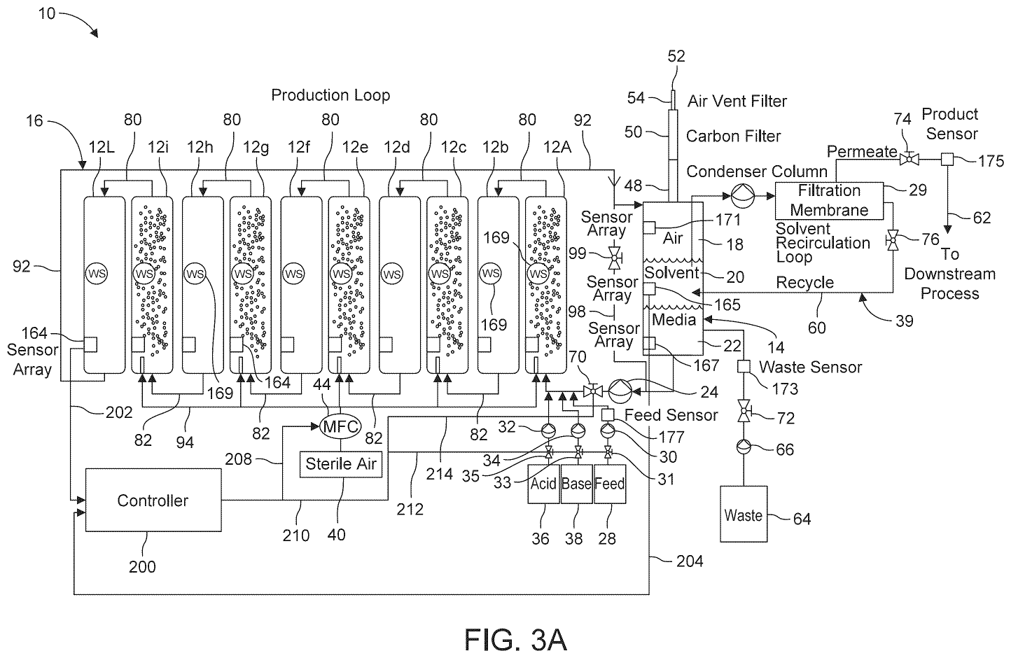

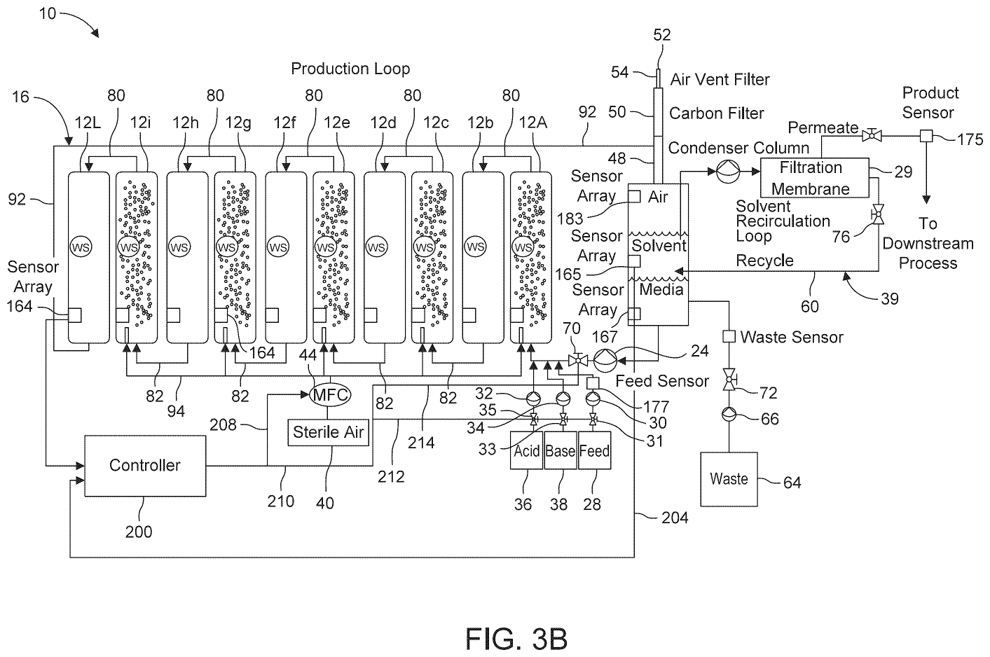

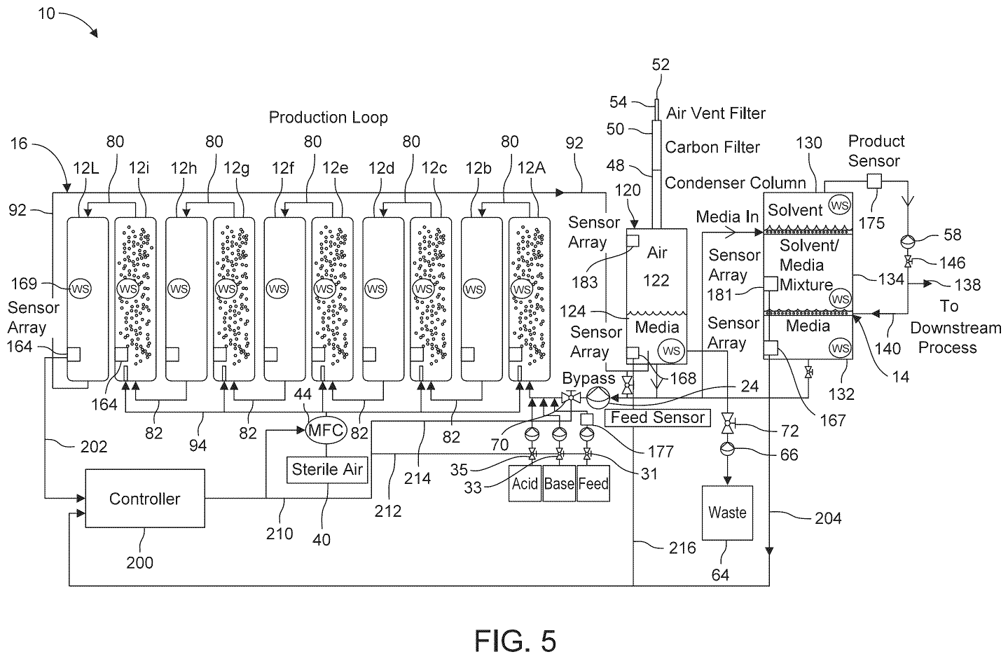

In a different approach, illustrated in FIGS. 3A, 3B, and 5, the synthesis vessels in production module 16 are arranged in series, for continuous operation, with aeration being supplied to select columns. In more detail, ingredients such as media from media reservoir layer 22 (of extraction vessel 14), supplemental media from reservoir 28, recycled media via loop 98 and valve 99 in the open position, acid from reservoir 36 and/or base from reservoir 38 are provided to the bottom of a first column, column 12A.

Contents extracted from the top of column 12A are transferred to the top of column 12b via conduit 80. Contents from column 12b are transferred from the bottom of column 12cb to the next column, column 12c, via conduit 82. The pattern continues from one column to the next, via conduits 80 and 82, until reaching the last column, column 12L.

From the bottom of this last column, bioreactor contents are directed to the extraction module through conduit (line) 92. In the embodiment of FIG. 3A, delivery is to gas layer 18, while in the embodiment of FIG. 3B, line 92 connects to condensation column 48 at the top of vessel 14.

Gas, typically sterile air, pure oxygen or a precise mixture of oxygen and nitrogen, from source 40 is supplied to the bottom of selected columns, e.g., columns 12A, 12c, 12e, 12g and 12j, using manifold 94. In some cases, the time that cells are without active aeration can be minimized by making the columns that do not receive air (e.g., columns 12b, 12e, etc.) more narrow than the columns with aeration (e.g., columns 12A, 12c, etc.)

Each column in production loop 16 is provided with sensors 164 (hard wired or wireless), attached at the bottom of the vessel, and sensors 169, which can be circulating with the circulating fluids within the vessel (or through the system or portions thereof) and are typically wireless.

The controller 200 receives data from sensors such as sensors 164 in the columns of production module 16 and (input 202) and/or from sensors in the extraction vessel 14, sensors (or sensor arrays) 165 and 167, for instance. As a result, (mass) flow controller 44 can be activated or adjusted, allowing more or less gas to the vessels. Valves such as valve 70 or reservoir valves 31, 33, 35, can be controlled via controller outputs 214 or 212. In one example, sensor 183 (in extraction vessel 14) is a level height sensor, such as an optical line break sensor, to detect foam generation.

Sensors (or sensor arrays) such as 171 (monitoring the air in vessel 14), 173 (monitoring waste), 175 (monitoring product) and 177 (monitoring the feed from feed reservoir 28) provide controller 200 with additional information, for further action as needed.

For product extraction, cells withdrawn from a synthesis vessel such as column 12 in FIG. 1 or column 12L in FIGS. 3A or 3B are transported through the organic solvent phase as the biofilm suspension circulates through the extraction vessel under the control of the controller 200. The suspension and media cascade out of the top inlet of the extraction vessel, through gas layer 18, and into a hydrophobic extraction solvent, such as hexane, heptane, dodecane, oleic acid, to name a few. Below the solvent layer 20 is an aqueous layer, from which the solvent spontaneously phase separates. Because of the lower density of the solvent, the solvent phase remains on top of the aqueous phase, the latter forming layer 22. The mixture of media, cells, biofilm, and, if used, solid supports such as beads fall through the solvent phase due to gravity. As they transit through the solvent, hydrophobic products are extracted into the hydrophobic phase. The productivity/viability of the biomass is maintained through the extraction process due to the innate tolerance of the biofilm forming organism to hydrophobic organic solvents. After spontaneous separation of the aqueous phase and the solvent phase, the aqueous phase collects at the base of the extraction vessel (layer 22), which can serve as a reservoir from which the aqueous media can be pumped into the synthesis vessel (using, for example, pump 24).

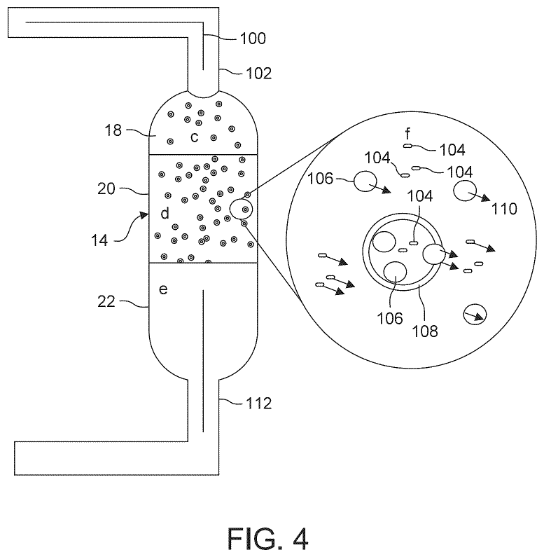

Processes taking place in extraction vessel 14 are illustrated in further detail in FIG. 4. With the cell suspension 100 being transferred from a synthesis vessel (e.g., reactor 12 in FIG. 1 or 12L in FIG. 3) to the extraction vessel 14, the aqueous mixture of biofilm (on supports or as self-aggregates), cascades from the extraction vessel inlet 102 through the air gap 18, then through solvent layer 20, settling into the reagent reservoir 22. Typically, the solvent layer 20 will contain: individual cells 104, cells in supported biofilm 106 (or in self-aggregated biofilm) as well as aqueous micelles 108, containing cells 104, cells in biofilm on supports 106 (or as self-aggregated unsupported biofilm). Contact between cells and solvent results in product 110 being transferred to (or extracted) into the solvent. The aqueous media from layer 22 can be withdrawn at outlet 112 and directed to one or more synthesis columns, as described above.

Shown in FIG. 5 is an arrangement that employs a production module 16 with columns in a series configuration (as described with reference to FIGS. 3A and 3B) and an extraction module including a set of vessels. In the embodiment of FIG. 5, contents from column 12L are directed to the bottom of a bubble trap 120, used to separate gas, e.g., air, shown in the upper region 122 of the bubble trap, from media occupying the lower region 124 of the bubble trap 120. The top of bubble trap 120 connects to condensation column 48 (for recapturing solvent) and carbon filter 50 (for preventing solvent leakage). Gas, e.g., air, gathered at the top region 122 of the bubble trap, is released at vent 52 which can be provided with a vent filter 54 (allowing the exhaust of sterile air). From the bubble trap 120, media (at the bottom region 124) can be directed to the bottom of column 12A (using pump 24 and opening valve 70), to waste by opening valve 72, or, with valves 70 and 72 closed off, to the upper region of extraction vessel 14, in this case a two-phase extraction column.

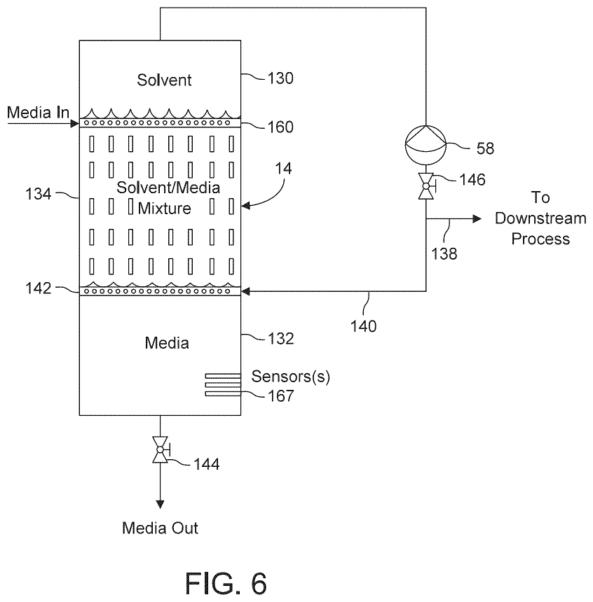

In the embodiment of FIG. 5, extraction vessel 14 no longer contains the gas layer 18 shown in FIGS. 1 and 3. Rather, as also presented in FIG. 6, the solvent forms an upper clarified solvent layer 130, clarified media settles as bottom layer 132, and, in between, a mixture of solvent and media forms intermediate layer 134. Solvent containing product is removed (using pump 58) from vessel 14 and can be collected or further processed, as generally shown by arrow 138. Solvent stream 140 can be returned to vessel 14, using, for example, distributor plate 142.

Another distributor plate, namely distributor plate 160 can be employed to introduce media to extraction vessel 14. Media that exits the bottom of vessel 14 can be directed to the bottom of column 12A, using pump 24 and valve 70.

In specific embodiments, distributor plate 142 and/or 160 are positioned at or near a fluid injection point (inlet), with the objective of lowering the disruption of the layer arrangement in the extraction vessel.

Extraction vessel 14 can be fitted with sensors 167, which can be placed at the bottom or on a wall of the vessel, or at another suitable location. Sensors 181 can be added to monitor the solvent-media mixture in intermediate layer 134. In the bubble trap, sensors 183 and 168 monitor, respectively, the air and the media.

Back pressure valves 144 and 146 control, respectively, flow through extraction vessel 14 back into the reactor system and through the solvent loop.

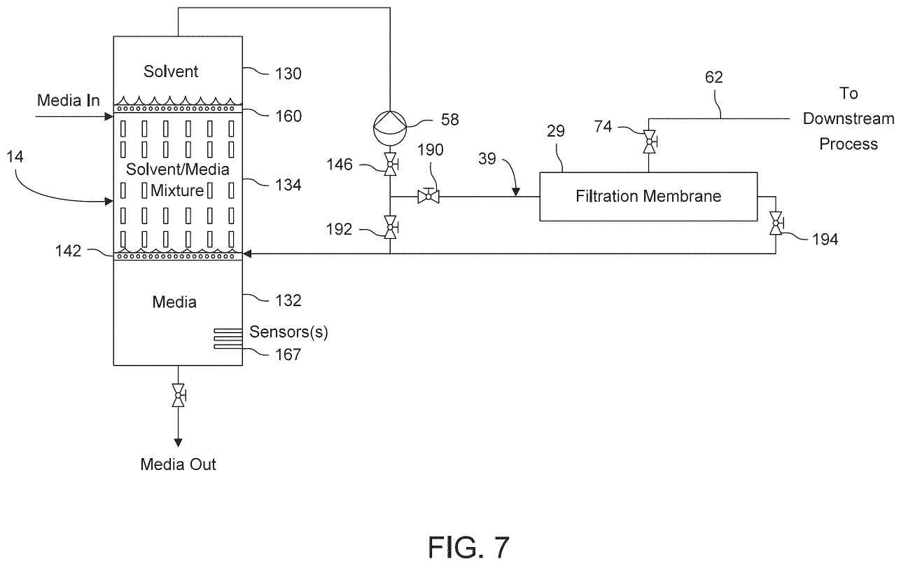

Shown in FIG. 7 is an arrangement in which a separation loop 39, e.g., a filtration loop (allowing the continuous harvesting of product from the system) is attached to the solvent circulation described with reference to FIG. 6. In this approach, pump 58 is controlled by the controller 200 to push the product-containing solvent, withdrawn from the top of vessel 14, through filtration membrane 29, where the two are separated. From outlet 62, product can be collected or directed to further processing equipment. Solvent can be returned to vessel 14 through distributor plate 142, for example. Valves such as valves 190, 192 and 194 control the flow of the various streams.

Flow controller 44 is controlled based on input 202 received by controller 200. Input 202 provides controller 200 with information from the sensors in the extraction vessel 14, while input 216 supplies data from the media sensor 168 in the bubble trap. In response (output 210, 212, 214) controller 200 will control pump 24, valve 70, valves 31, 33, 35, essentially as described above.

Sensors such as 164 and 169 (synthesis vessel), 167 and 181 (extraction vessel), 168 and 183 (bubble trap), and/or other sensors (e.g., sensors 171, 173, 175, 177) can be employed to enable the controller 200 to monitor the process parameters such as surface temperature, pressure, internal temperature, pH, DO, CO₂, and so forth. Some implementations provide electrochemical or optical sensors for tracking and quantifying the pH, CO₂, and O₂ or for carbon sources (e.g., acetate, lactate, succinate, glycerol, etc.) found in the bioreactor. In more detail, the sensors used can monitor biofilm health, system oxygenation, pH, and/or chemicals present in the feedstock or extraction solutions. In some examples, the controller 200 dynamically responds to sensor readouts to alter flow rates, adjust nutrient or gas concentrations, or notify the operator about a system failure or a contamination event.

In many cases, the sensors are in contact with media at the base of the reactor where they can be directly embedded in the reactor base or wall. Sensors that float in vessel (a reactor or an extraction vessel) or circulate throughout the system or portions thereof can be utilized, as can be removable sensors. It is also possible to sample the media by drawing it into a secondary sensing chamber. Aliquot extractions and offline analysis can be employed as well.

Information from sensors, pumps, valves, and reservoirs, can be used to alter or adjust media composition, e.g., by introducing more carbon, or by treatment with acid/base to adjust the pH. The controller 200 often operates autonomously executing a closed-loop system control algorithm.

In one aspect of the invention, the controller 200 is a computer (such as a single board computer) or other digital control system to control process parameters and/or system components (e.g., pumps, valves, the thermal system, etc.) and monitor the sensors. The computer processes the sensor inputs and sends signals to the thermal jacket to adjust the temperature, can open and close valves or adjust the rate of pumping to control the introduction of feedstock and/or extraction solutions, and can alert the operator to performance degradation, contamination, or clogging.

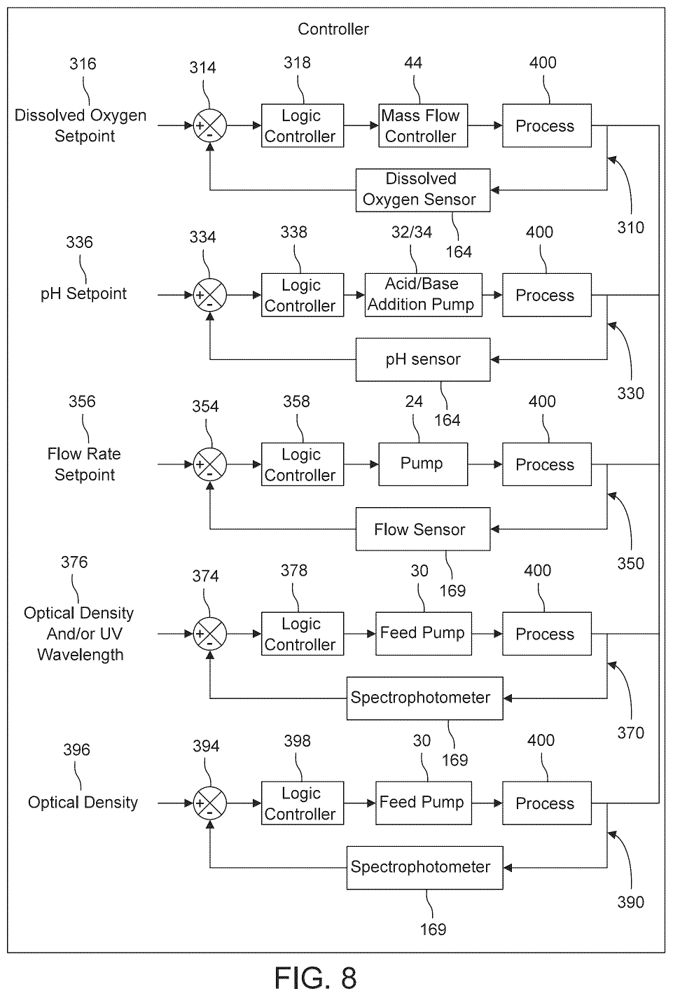

FIG. 8 is a block diagram illustrating processes 300 by which controller 200 addresses some sensor data. In loop 310, for example, a sensor (e.g., one of sensors 164) detects DO levels in a vessel and transmits the data to controller 200 for an assessment or comparison 314 to a DO set point (set value) 316. Logic controller component 318 controls flow controller 44 (to add air or to cut back on the air supply, for example), thus adjusting parameters in the overall process 400, which runs continuously.

In some examples, the logic control components are threads running a processor or can be a separate hardware systems or subsystems.

In pH loop 330, a pH sensor (one of sensors 164, for instance) transmits data that is compared (step 334) to a set point 336. Acid or base can be added from reservoirs 36 or 38 (by opening valves 35 or 33 and running pumps 32 or 34) in response to logic controller component 338, thus optimizing continuous process 400.

A flow rate loop 350 received process flow rate readings from one or more sensors such as, for instance, a wireless sensor 169 (in one of the synthesis columns in FIGS. 3A, 3B or 5) or a sensor circulating through various system components. As a possible result of comparison 354 to a flow rate set point 256, logic controller component 358 can adjust a pump (e.g., pump 24), and thus the flow rate of the overall process 400.

In the chemical analysis (optical density and/or ultraviolet (UV) wavelength) loop 370, a sensor such as one of the wireless sensors 169 (or a spectrometer) supplies chemical composition information for a comparison (step 374) with a set point value 376. Based on input from logic controller component 378, feed pump 30 is turned on or off, thus optimizing the overall process 400. In addition, the logic controller can set the circulation rate, aeration, waste rate, solvent circulation rate, and solvent removal rate, each of which can be adjusted by this control loop.

Loop 390 depicts an optical density loop in which information from one of the wireless sensors 169 (or a spectrometer) supplies data that is compared (step 394) with an optical density set value 396. Based on input from logic controller component 398, feed pump 30 can be opened or closed, opening or shutting off media supply from reservoir 28, thus optimizing the overall continuous process 400. In some embodiments, the logic controller sets the circulation rate, aeration, waste rate, solvent circulation rate, and solvent removal rate, each of which can be adjusted by this control loop.

As seen in the illustrative drawings discussed above, many embodiments of the invention feature reservoirs, conduits, pumps, valves, flow controllers, sensors, and a computer control system. Solutions are routed among these components using fluidic channels. Mixers, arrangements for introducing gas, output analysis, a phase separator, and/or a product purification module also can be present. One or more vents for releasing pressure build-up can be provided in the synthesis module, in the extraction module or in both.

Equipment to control the introduction and removal of fluids, for example, may include suitable containers for liquids, fluidic channels (such as tubing, pipes, joints, and the like), valves, pumping systems, flow sensors, computer control of fluid flows, and so forth. In some cases, a configuration employing gravity may eliminate the need for one or more pumps.

As noted above, various devices (diffusers, spargers, distributor plates, etc.) can be employed to provide desired conditions for the introduction of fluids (whether in liquid or gaseous form) into a vessel, e.g., a bioreactor and/or extraction vessel. In some embodiments, the system incorporates devices that promote mixing. Specific examples utilize such devices to enhance fluidization and contact among the materials in the synthesis vessel. The mixing device can be placed at a desired location, often at the bottom of the reactor, for instance at the inlet for feeding media to the bioreactor.

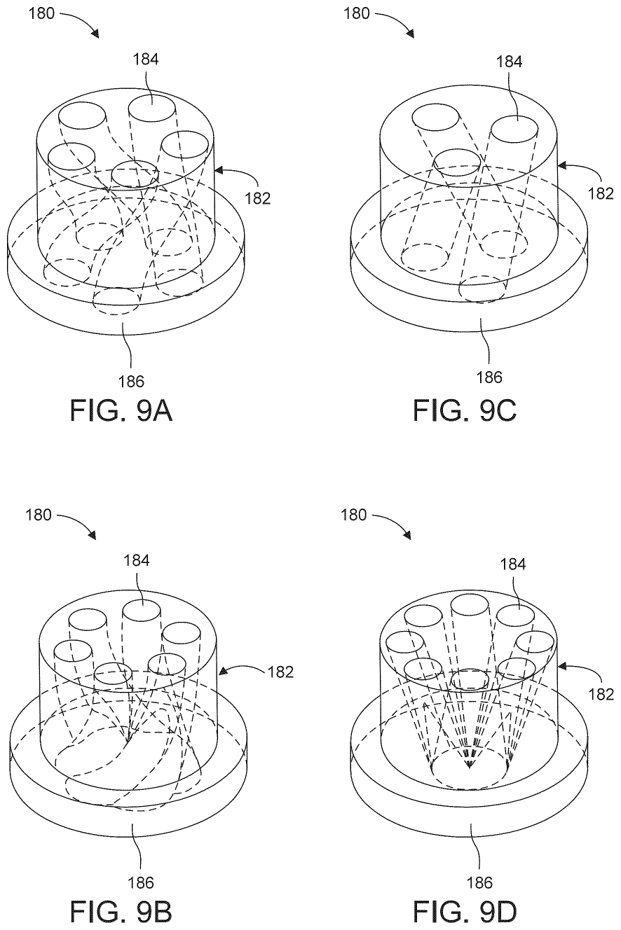

Designs of a mixer or vortex generator that can be employed are presented in FIGS. 9A through 9D. Shown in these drawings is mixer or vortex generator 180, including a mixer body 182, made from a suitable material such as, for instance, plastics, metal, fiberglass, ceramic, to name a few. The body 180 defines multiple channels 184. The channels can be formed by drilling, e.g., laser drilling, or another suitable technique, as known in the art. In one example, mixer 180, including channels 184, is fabricated by 3D printing with PETG (polyethylene terephthalate glycol-modified).

In FIGS. 9A and 9B, the channels are curved, while the designs of FIGS. 9C and 9D include straight channels 184. In all cases, the channels are not parallel to an axis that is perpendicular to base 186 of mixer body 182. Rather, they are slanted or angled, often outwardly. The angular flow can enhance mixing efficiency, increase flushing speed, reduce or minimize jamming, and/or lower the pumping requirements.

Mixer 180 can be dimensioned according to vessel or process requirements. In one example, block 182 has a height within a range of from about 6 in to about12 in, and a largest diameter within a range of from about 6 in to about 24 in. The diameter of 182 will be matched to the internal diameter of the bioreactor vessel. In many cases, all channels 184 have the same shape, diameter and/or slant angle. In one illustration, the channels have a length within a range of from about 6 in to about 24 in, and a diameter within a range of from about 0.25 in to about 6 in. The height of block 182 is determined by the distance needed to create an appropriate fluidic vortex for a particular pipe diameter.

Heating or cooling can be provided by elements such as heat exchangers, heating tape, thermoelectric elements, peltier cooling elements, or others, as known in the art. In one example, vessels are wrapped in heating jackets. Temperature sensors can signal the need for temperature adjustments, and the heating element can be turned on or off automatically.

The synthesis/extraction cycle can be operated continuously for weeks or months until the reactor productivity degrades or the reactor becomes contaminated. Semi-continuous or even batch schemes can be implemented in some cases.

### Genetically-Engineered Microorganisms Suitable for Use in the Circulating Biofilm Reactor

In general, the microorganism employed to form the biofilm) on slid supports or as self-aggregates, is selected based on its innate mechanisms for converting a carbon source to a desired product, often a hydrophobic chemical. Examples of products that can be bio-manufactured using techniques described herein include but are not limited to isoprenoids, carotenoids, retinoids, or other substances. In many embodiments, the organism is tolerant to organic solvents, a property that enables two phase extraction of products while preserving functional biomass.

A circulating biofilm reactor for producing isoprenoids, carotenoids and retinoids as described herein comprises suitable biofilm-forming, solvent-tolerant, hydrocarbonoclastic organisms. Specifically, the engineering of a hydrocarbonoclastic organism (also known as hydrocarbon degrading bacteria) with a pathway to produce isoprenoids and retinoids can enable more efficient methods of biological isoprenoid and retinoid synthesis through direct extraction using organic solvents, for example, using a biofilm or biofilm reactor according to the present invention.

These genetically-engineered hydrocarbonoclastic organisms are selected from species of prokaryotes or archaea which can degrade and utilize hydrocarbon compounds as a source of carbon and energy. As described herein these hydrocarbonoclastic organisms, are used in methods to produce (biosynthesize) a class of compounds called isoprenoids or terpenoids (terpenoids/isoprenoids are organic compounds derived from the 5-carbon compound-isoprene and isoprene polymers, terpenes). Degrading and utilizing hydrocarbons are a characteristic of hydrocarbonoclastic organisms such as Marinobacter spp. (Gauthier, 1992; Handley, 2013) or Pseudomonas spp. (Isken, 1998).

Specifically encompassed are hydrocarbonoclastic microorganisms that are genetically-engineered for increased biological activity relative to its wild-type organism to synthesize/produce isoprenoids, carotenoids, or retinoids (e.g., wherein the product molecule/compound is e.g., retinal or retinol) in high yield in a biofilm or biofilm bioreactor. Such genetically-engineered microorganisms, as well as their nucleic acid sequences, are described in detail in U.S. patent application Ser. No. 17/722,182 (hereinafter the '182 application), the teachings of which are incorporated herein by reference.

Hydrocarbononoclastic microorganisms suitable for use in the present invention have, inter alia, two important characteristics: the ability to form a stable biofilm (e.g., in a biofilm reactor) and a tolerance to hydrophobic organic solvents. Such microorganisms include, for example, Marinobacter species, and Pseudomonas species. More specifically, encompassed by the present invention are biofilm-forming hydrocarbonoclastic microorganisms, such as Marinobacter spp., and in particular Marinobacter atlanticus, that are capable of forming biofilms and have tolerance to hydrophobic organic solvents. Such hydrocarbononoclastic organisms are genetically-engineered to contain one, or more nucleic acid or amino acid sequence variations/mutations in one, or more (e.g., a plurality of) genes that make up the mevalonate and/or carotene synthetic pathway. Nucleic acid (DNA) that encode for the genetically-engineered operons and/or genes in the mevalonate, beta-carotene, and retinol pathways and are engineered, for example, through codon harmonization for expression in Marinobacter spp. DNA sequences for exemplary genetically-engineered microorganisms are, for example, SEQ ID NOS: 1-30 as shown in FIGS. 1-30 of the '182 application. These sequences are also incorporated in the present application as indicated above.

The operon is responsible for gene expression and protein synthesis in prokaryotes. An operon, as described herein, is a grouping of (or a region of) one, or more related genes/gene sequences which are expressed to produce one, or more biologically active enzymes/proteins. The operon comprises one, or more, gene sequences encoding the desired proteins, a promoter sequence and an operator sequence (the operator sequence can be located within the promoter sequence or as a separate sequence). The operon is responsible for the transcription of DNA into messenger RNA (mRNA) which is then translated into the desired protein(s) or enzyme product in the prokaryote.

As described herein, the present invention encompasses variant operons/genes that encode enzymes/proteins having biological activity that differs from its counterpart wild-type (non-altered) operons/genes. One example of increased biological activity of the variant operon/gene over its wild-type counterpart is to increase the yield of the desired product such as the retinoid compound. Another biological activity described herein is the ability/capability to form a more stable biofilm, for example in a bioreactor. Yet another biological activity described herein is increased stability to organic solvents. One example of increased biological activity of the variant operon/gene over its wild-type counterpart is to allow the expression of the necessary genes and subsequent enzymes in a hydrocarbonoclastic/oleaginous biofilm forming organism.

For example, in some embodiments of the present invention, the variant genes encode genetically-engineered promoter sequences to increase expression of the desired enzymes, thus resulting in increased synthesis, yield or stability of the desired retinoid compound. In another example, the specific genes comprising an operon can be rearranged resulting in a variant operon that differs in biological activity from the wild-type operon, again resulting in increased synthesis, yield or stability of the desired retinoid product.

More specifically encompassed by the present invention, is a genetically engineered (also referred to herein as genetically modified or altered) hydrocarbonoclastic microorganism wherein the operon genes encoding enzymes required for mevalonate production pathway have been optimized by codon harmonization (also referred to herein as synthetic genes), wherein the pathway is designed to route the native acetyl-CoA pool of Marinobacter spp. or a similar hydrocarbonoclastic organism to the production of isoprenoids such as retinal or retinol. Such microorganisms can be further modified to include variant genes/operons of the beta-carotene synthetic pathway designed to route beta-carotene to the production of retinal, retinoate, retinol or retinyl esters.

As described herein, these enzymes (also referred to herein as variant proteins or synthetic enzymes) from these pathways will be engineered to improve performance, that is, the nucleotide sequences that encode for these modified enzyme sequences will be optimized (e.g., by codon harmonization, mutations, insertions or alterations resulting in the variant enzymes differing in sequence and biological activity from their wild-type/naturally-occurring counterpart enzyme) to alter/modify (typically increase) the biological/catalytic activity of the enzymes as compared to (i.e., relative to) enzymatic activity encoded by the operon genes in the wild-type (unmodified) microorganism. Methods of genetically engineering the microorganisms are described herein and methods of evaluating enzymatic/biological activity of proteins are also described herein and are known to those of skill in the art.

The genetically engineered microorganisms include/comprise the specific arrangement of the plurality of genes for retinal or retinol synthesis into an operon in which the genes are arranged in a manner such that enzyme expression is optimized for highest yield product synthesis.

Specifically described herein are organisms comprising variant genes in the mevalonate synthetic pathway (also referred to herein as the MVA pathway), encoding enzymes that convert acetyl-CoA into isopentenyl pyrophosphate (IPP), which is the building block of all isoprenoids (U.S. Pat. No. 7,172,866 B2, the teachings of which are incorporated herein by reference in their entirety). Such variant genes include the nucleic acid Sequences 5, 6, 7, 8, 9, 10 or 15, (SEQ ID NOS: 5, 6, 7, 8, 9, 10 or 15) or sequences comprising about 80, 85, 90, 95, 96, 97, 98 or 99% sequence identity to Sequences 5, 6, 7, 8, 9, 10 or 15 of the '182 patent application.

In some embodiments, the organism is also genetically-engineered with one, or more, additional variant genes introduced into the organism wherein such genes make up the beta-carotene pathway, which encodes enzymes that convert IPP to beta-carotene. (See FIG. 32). Such variant genes include the nucleic acid Sequences 11, 12, 13, 14, 15, 17, 18, 19, 20, 21, 27, 28 or 29 (SEQ ID NOS: 11, 12, 13, 14, 15, 17, 18, 19, 20, 21, 27, 28 or 29), or sequences comprising about 80, 85, 90, 95, 96, 97, 98, or 99% sequence identity to Sequences 11, 12, 13, 14, 15, 17, 18, 19, 20, 21, 27, 28 or 29 of the '182 patent application.

In one embodiment the genetically-engineered organism comprises introduction of a variant blh gene encoding the 15,15′-dioxygenase, (Sequence 16 of the '182 patent application) wherein the introduction of the variant blh gene results in the production of retinal and/or retinol.

Also encompassed by the present invention is a variant human retinol

dehydrogenase 12 (RDH12) gene encoding the retinol dehydrogenase comprising Sequence 30, and its encoded protein comprising Sequence 30. The retinol dehydrogenase gene (RDH12) can comprise a nucleic acid sequence selected from the group consisting of: Sequence 17, Sequence 18; or Sequence 20 (SEQ ID NOS: 17, 18 or 30), or a sequence comprising about 80, 85, 90, 95, 96, 96, 98, or 99% sequence identity to Sequences 18, 19 or 20. The encoded RDH12 protein can also encompass sequences comprising about 80, 85, 90, 95, 96, 97, 98, or 99% sequence identity to SEQ ID NO: 30, wherein the protein has aldehyde dehydrogenase biological activity comparable to the variant RDH12 activity. Further encompassed by the present invention are genetically-engineered organisms wherein the organism comprises introduction of a variant RDH12 gene (SEQ ID NOS: 17, 18 or 20) expressing a variant retinol dehydrogenase 12 (RDH12) SEQ ID NO: 30), wherein the introduction of the variant RDH12 gene results in the conversion of retinal to retinol. These sequences are listed in patent application '182.

Also encompassed is a genetically-engineered organism, wherein the organism comprises introduction of a variant ybbO gene (SEQ ID NOS: 19 or 21), wherein the introduction of the variant ybbO gene results in the conversion of retinal to retinol.

Also encompassed herein are genetically-engineered organisms comprising the variant operon sequences described herein. For example, the organisms of the present invention can comprise a variant operon of the upper mevalonate pathway comprising Sequence 1 (SEQ ID NO: 1) and/or a variant operon of the lower mevalonate pathway comprising Sequence 2 (SEQ ID NO: 2).

The genetically-engineered organisms of the present invention can further comprise variant operon sequences of the beta-carotene pathway, wherein a variant operon sequence is selected from the group of sequences consisting of: Sequence 3; Sequence 4; Sequence 22; Sequence 23; Sequence 24 or Sequence 25 (SEQ ID NOS: 3, 4, 22, 23, 24 or 25).

One particular embodiment comprises the genetically-engineered organism, wherein the variant mevalonate pathway gene(s) comprise the variant operons of SEQ ID NO: 1 and SEQ ID NO: 2, and the variant carotene pathway gene(s) comprise the variant operons of SEQ ID NO: 3 and SEQ ID NO: 4. Another particular embodiment comprises the genetically-engineered organism, wherein the variant mevalonate pathway gene(s) comprise the variant operons of SEQ ID NO: 1 and SEQ ID NO: 2, and the variant carotene pathway gene(s) comprise the variant operons of SEQ ID NO:22 and SEQ ID NO: 26.

All nucleic acid sequences and amino acid sequences described herein include sequences with sequence identities of about 80, 85, 90, 95, 96, 97, 98, or 99% sequence identity to the described sequences. Such sequences will have comparable biological activity (essentially the same within a few measures of activity) as the described sequence when evaluated using standard techniques.

In some embodiments of the present invention, these genes/operons are introduced into an expression vector, such as a plasmid, suitable/compatible for expression of the genes in a competent host cell. Specifically, the host as described herein, is a hydrocarbonoclastic microorganism, specifically a Marinobacter species organism, and more specifically a Marinobacter atlanticus microorganism. After introduction of the expression vector comprising the variant genes of the present invention, under suitable conditions well known to those of skill in the art, the variant genes are incorporated/inserted into the genome of the host organism for expression. Techniques of genetic transfer into cells are known to those of skill in the art. Also encompassed by the present invention are host cells comprising the vectors or plasmids described herein.

In some embodiments, the hydrocarbonoclastic organism produces isoprenoids, carotenoids, or retinoids from aromatic or aliphatic molecules. In yet other embodiments the hydrocarbonoclastic organism produces isoprenoids, carotenoids, or retinoids from short chain fatty acids. In some of these embodiments, the short chain fatty acid is lactate from dairy waste.

The present invention further encompasses a biofilm comprising a genetically-engineered hydrocarbonoclastic microorganism as described herein, and its use in the biofilm reactor as described herein.

Also encompassed by the present invention are methods for the production/synthesis of isoprenoids, carotenoids, and retinoids using a genetically engineered hydrocarbonoclastic organism such as Marinobacter species, or Pseudomonas species as described herein, in a biofilm or biofilm bioreactor. Specifically encompassed by the methods described herein is the production of the isoprenoids beta-carotene, retinal, retinol or squalane. Importantly, these methods comprise the use of organic solvents (e.g., non-polar solvents) to extract the isoprenoids without significant, or substantial, degradation of the isoprenoid product, resulting in higher yield, synthesis and/or stability of the desired product. For example, the synthetic biofilm and biofilm bioreactor described herein, and methods of synthesizing isoprenoids and retinoids can comprise the use of hexanes, dodecane, or oleic acid as an extraction solvent of the desired product can be determined by techniques known to those of skill in the art.

In some embodiments, the extraction solvent specifically contains an anti-oxidant or an encapsulant to prevent oxidation or degradation of the product. For example, the extraction solvent can include a molecule such as cyclodextrin to stabilize the product molecule. In other embodiments, lipid molecules dispersed in solvent microdroplets are used to simultaneously extract the product and encapsulate the product in a liposome.

In a specific embodiment of the present invention, the method encompasses the production/synthesis of an isoprenoid used as an ingredient or component in the formulation of a cosmetic product, wherein the cosmetic ingredient is substantially free of contaminants that can be found in the isoprenoid produced by conventional methods. For example, the product retinol is often used in cosmetic creams or ointments manufactured for human use, so the purity of the retinol is extremely important. Evaluation of the purity, or degree of contamination, of lack of contamination, of the end product can be performed by methods known to those of skill in the art. Specifically, the product of the methods described herein is a cosmetic ingredient suitable for veterinary use or human use (e.g., retinol in a cosmetic facial cream) and the extraction solvent of the method is a component or a suitable additional ingredient of the cosmetic preparation/formulation. For example, the cosmetic ingredient can be an emollient, and in one embodiment the emollient is squalane.

Embodiments of the invention are illustrated in the following non-limiting examples.

### EXAMPLE 1

### Synthesis of Retinol in Bioreactor

The bioreactor is constructed by stacking 2×6 inches (″) diameter×20″ high sight glasses, which connect to each other using tri-clamp fittings to form the synthesis vessel. The synthesis vessel is connected using 0.5″ stainless piping via an outlet port at the top of the sight glasses to a third 6″×20″ sight glass, which serves as the extraction vessel. An air driven pump connects the base of the extraction vessel to inlets on the perimeter of the base of the synthesis vessel. The synthesis vessel contains a center inlet at its base that houses a carbonation stone. The reactor contains ˜30 L of total media and is loaded with between 1 and 20 lbs of glass beads, which serve as the solid support for the biofilm. These beads can be pre-seeded with retinol-producing cells or liquid culture of planktonic retinol-producing cells, which are introduced into the bioreactor to seed the growth of the biofilm. The reactor is circulated for between 12 and 24 hrs to establish the biofilm, at which point between 100 mL and 10 L of hexane, heptane, or dodecane is added into the extraction vessel. The biofilm suspension is circulated through the bioreactor at a rate of between 0.1 and 10 L/min. The product is continuously extracted into the solvent phase and collected from the separation module.

### EXAMPLE 2

### Use of Vortex Generators

Inducing angular flow into the bioreactor can help promote better mixing at lower pumping speeds. To achieve this angular flow, vortex generators are 3D-printed with polyethylene terephthalate glycol (PETG) and placed in the base of each individual column of the bioreactor. Vortex generators can be printed with PP or another material to allow them to be steam sterilized in place. In initial experiments, these inserts were seen to improve flushing speed of the reactor by an order of magnitude or more.

### EXAMPLE 3

### Wireless Sensors for Controlling Bioreactor Operation

Small, pill size wireless sensors (such as those described in, “A Threshold-Based Bioluminescence Detector With a CMOS-Integrated Photodiode Array in 65 nm for a Multi-Diagnostic Ingestible Capsule”, Liu, Q., Jimenez, M., Inda, M.E., Riaz, A., Zirtiloglu, T., Chandrakasan, A.P., Lu, T.K., Traverso, G., Nadeau, P. and Yazicigil, R.T., 2022. A Threshold-Based Bioluminescence Detector With a CMOS-Integrated Photodiode Array in 65 nm for a Multi-Diagnostic Ingestible Capsule. IEEE Journal of Solid-State Circuits, 58(3), pp.838-851.) are distributed throughout the bioreactor system. Sensors are both fixed in place inside the reactor system, or actively circulating along the flow path with the cells through the reactor system. These sensors use optical signals as described in Liu et al., or electrochemical signals to measure the state of the bioreactor at a particular location and a particular moment in time. The specific optical or electrochemical signals correspond to measurements of pH, carbon dioxide, oxygen, the level of feedstocks components such as glycerol, lactate, acetate, or other organic acids, glucose or other sugars An electrochemical fingerprint (cyclic voltammetry and/or electrochemical impedance) from these sensors provides a snapshot of the bioreactor state (e.g., cell density, cell health, available feedstock)

At process startup, the reactor is filled with sterile media and inoculated. Air flow into the columns is initiated and oxygen saturation is monitored on different sensors. The wireless sensors inside the bioreactor communicate the instantaneous oxygen saturation to a wireless receiver connected to a microprocessor. This microprocessor uses a PID algorithm to determine the appropriate air flow setting for each column and sends signals to mass flow controllers or to proportional valves (I/P or E/P convertors) that convert a variable current or voltage signal into a proportional compressed air output. Typical ranges for dissolved oxygen are between 1 and 9 mg/L, with the specific setpoint relating to the desired growth rate and redox environment. The cell density is continuously measured at different points within the reactor either using electrochemical impedance measurements, which vary due to increasing biomass altering the dielectric properties of the reactor environment or optical density measurements which change due to increased scattering that corelates to increased cell density. These data are wirelessly transmitted to a microprocessor type controller which processes the information together with the information about oxygen consumption to control the introduction of fresh feedstock into the reactor. To introduce fresh feedstock, the microprocessor sends a digital signal to open a feedstock control valve, which could be a ball valve or a solenoid valve. The microprocessor then sends a digital signal to turn on a pump at the flow rate specified by the algorithm to introduce additional feed.

Once the desired biomass density has been achieved, the microprocessor switches to continuous operation mode. In this mode, feed is continuously introduced at a specified rate. Sensors continue to be constantly monitored along the reactor. The electrochemical measurements made in each column are transmitted to the microprocessor, which uses these measurements to assess cell health and productivity. Depending on the electrochemical signature, the reactor will either (1) increase or decrease the feed rate, (2) adjust pH, (3) introduce a nutrient feed, (4) alter air flow, (5) alter the circulation flow rate, (6) carry out a controlled dilution in the reactor by sending some media to waste and introducing fresh feedstock. If the measurements indicate contamination or are outside the bounds of normal readings, the microprocessor will send a message to the operator by email, text message, or otherwise indicate a problem by illuminating warning lights or sounding an alarm.

Sensors located in the extractor measure product concentration and quality either optically or electrochemically. The signal is wireless transmitted to a microprocessor which analyzes the data. For example, for retinol as a target product, optical absorption spectroscopy sensors can measure absorbance at the optical wavelengths 325 nanometer (nm), 350 nm, and 368 nm. The microprocessor controller uses the ratio of these signals to determine the relative concentrations of retinol, retinal, and retinoic acid. If the concentration of retinoic acid is too high, the microprocessor can adjust the oxygen concentration in the final reactor stages to create a more reducing environment or increase the overall flow rate so that product is removed with higher frequency. If the concentration of total retinoids is too high, the sensor can alter the flow rate from the extractor into the nanofiltration system.

### EXAMPLE 4

### A Multicolumn Bioreactor Run in Parallel for the Production of Hydrophobic Proteins

A bioreactor system, as depicted, for example, in FIG. 1 is configured with 10 columns operating in parallel, feeding into a single extractor. Media is introduced into the reactor and it is inoculated with a strain of Marinobacter engineered to highly express a hydrophobic protein and export it from the cells. The cells are grown to a cell density of between an OD600 equivalent of between 5 and 30 and then maintained at this cell density using the microprocessor which monitors the sensors and control the feed, aeration, the introduction of nutrients or acid and base solutions to balance pH, and wasting of media. The sensors in each column are continuously monitored to ensure balanced flow throughout the system, consistent cell densities and feeding rates, and to identify any drops in cell vitality or potential contamination events.

The cells, biofilm, and media (feedstock) are all continuously pumped upwards through each column and circulated through the extractor containing the organic solvent. The media falls through the organic solvent due to gravity, and then is pumped back into the reactor. Maintaining the media balance in the extractor is important to ensuring that the solvent remains in the reactor. This is achieved by using sensors to continuously monitor the solvent levels in the reactor and balance the media level. During initial biomass accumulation and protein maturation stages during the initiation of a run the extractor can by bypassed using a valve to allow all of the media or a fraction of the media to skip the extractor. A level height sensor, such as an optical line break sensor, in the air portion extractor monitors for foam generation. Should the media/solvent/foam level get too high, the line break sensor will signal the microprocessor controller, which will then turn down or off the pump and/or the aeration to allow the foaming to decrease. As the cells produce protein it is excreted from the cells and accumulates in the solvent layer as the cells and media are washed through the extractor. The solvent is continuously circulated through a filtration membrane by the controller, which provides size-selective extraction of the protein of interest. In some embodiments a multi-staged membrane process may be used where the first membrane has a smaller size than the target molecule and a second membrane has a larger size than the target molecule. In the first stage, the permeate would be returned the reactor by the controller 200, and the retentate sent to the second stage, this will concentrate the protein in the solvent. In the second state, the protein will pass through the membrane, eliminating any cellular debris or larger proteins, while the retentate would be returned to the reactor. The product can be collected for further purification such as affinity tag purification or other methods of chromatographic separation.

The process is designed to operate continuously until sufficient protein is produced or the productivity of the cells decreases.

### EXAMPLE 5

### A Multi Column Bioreactor Run in Series for the Production of Retinoids

A bioreactor system, as depicted in FIGS. 3A or 3B, is configured with 10 columns operating in series and feeding into a single extractor. This system can alternatively have an air trap prior to the extractor, as shown in FIG. 5, to allow pressure to leave the system prior to extraction, which can reduce emulsification in the extractor.

FIGS. 3A, 3B and 5 show a configuration of the bioreactor where columns are paired (one column in each pair receiving aeration and the other being without aeration) where media is pumped up through one column and then down through a second. In some embodiments, the columns without aeration may be more narrow than the columns with aeration, effectively pipes that connect one reactor to the next, thus minimizing the time the cells experience without active aeration.

At process startup, the reactor is filled with sterile media and inoculated with a retinoid producing strain of Marinobacter. The cells can optionally be circulated through the reactor without entering the extractor during startup to achieve the desired biomass. The biomass sensors monitor biomass, and once the target biomass is achieved as determined by the controller 200, typically at least OD600 equivalent of at least 1-5, the cells are circulated through the extractor. On each cycle through the columns, feedstock is introduced by the controller 200, cells convert the feedstock into product as they pass through each column, finally reaching the extractor. In the extractor the aqueous solution is dispersed in the immiscible solvent and falls through the solvent due to gravity, extracting the product. Each cycle, a fraction of the aqueous phase is removed from the reactor by the controller 200 to allow for the addition of more feedstock and maintain a constant fluid level.

To achieve optimal operation, the residence time of the cells in the series of columns and the feed rate can be balanced to achieve a target feed consumption over the course of circulation through the reactor, for example, consumption of 50% of the feed at a flow rate of 1 L/min or 90% of the feed at a flow rate of 0.1 L/min. The microcontroller can monitor sensor signals in real time to assess product production via optical signals in the extractor, and quantify feedstock consumption in each column based on the wireless sensors in each of those columns. The microcontroller can then adjust the feed and flow rates to ensure that the target feedstock consumption is achieved during each cycle.

The biosynthetic pathway for retinol production includes 14 steps with 13 genes, the final steps of which require reducing conditions. The multicolumn design of the bioreactor system allows the tuning of the reactor circulation to match the kinetics of retinoid production and operate the reactor with different zones—an oxygen rich zone for initial production of acetyl-CoA, critical coenzymes such as NADH/NADPH, and early pathway precursors, followed by a more reducing zone where the conversion of the final product can take place. The sensors in each column measure the oxygen level and characterize the local redox environment electrochemically. This signal is then analyzed by the microprocessor, which can turn the air pressure up or down in individual columns to create the desired oxygenation profile.

Once the retinoid product is extracted into the solvent, it is circulated through the nanofiltration system to concentrate and size-selectively purify the retinol from the other cellular components. Through the appropriate choice of nanofiltration membranes, there is the ability to selectively purify retinol, retinoic acid, or retinal.

While this invention has been particularly shown and described with references to preferred embodiments thereof, it will be understood by those skilled in the art that various changes in form and details may be made therein without departing from the scope of the invention encompassed by the appended claims.

What is claimed is:

1. A circulating bioreactor system for producing chemical products, comprising:
one or more synthesis vessels, configured to support the growth of microorganisms and facilitate the conversion of a carbon source into a desired product;
an extraction vessel, configured to extract the desired product from the media into an organic solvent;
a controller for the bioreactor system; and
wireless sensors, operably deployed within the bioreactor system to monitor process parameters, the wireless sensors being configured to transmit data of the process parameters to the controller.

2. The circulating bioreactor system of claim 1, wherein the wireless sensors are configured to monitor one or more process parameters selected from the group consisting of pH, dissolved oxygen, carbon dioxide concentration, temperature, pressure, flow rate, and feedstock composition.

3. The circulating bioreactor system of claim 1, wherein the wireless sensors are further configured to measure biomass density and biofilm health within the synthesis vessels.

4. The circulating bioreactor system of claim 1, wherein the wireless sensors are configured to monitor solvent levels and product concentration in the extraction vessel.

5. The circulating bioreactor system of claim 1, wherein the wireless sensors are distributed throughout the bioreactor system to monitor conditions in multiple locations, including the synthesis vessels, extraction vessel, and connecting conduits.

6. The circulating bioreactor system of claim 1, wherein the wireless sensors are configured to circulate within the bioreactor system along with the media flow to provide spatially distributed measurements.

7. The circulating bioreactor system of claim 1, wherein the wireless sensors include optical sensors configured to measure process parameters through fluorescence or absorbance spectroscopy.

8. The circulating bioreactor system of claim 1, wherein the wireless sensors include electrochemical sensors configured to perform cyclic voltammetry or electrochemical impedance spectroscopy for monitoring process parameters.

9. The circulating bioreactor system of claim 1, wherein the wireless sensors are configured to transmit data in real time to the controller, enabling dynamic adjustments to process parameters.

10. The circulating bioreactor system of claim 1, wherein the controller uses data from the wireless sensors to adjust gas flow rates, nutrient feed rates, or circulation flow rates within the bioreactor system.

11. The circulating bioreactor system of claim 1, wherein the wireless sensors are configured to detect deviations from normal process parameters for the controller.

12. The circulating bioreactor system of claim 1, wherein the wireless sensors are configured to monitor redox conditions and oxygen levels to enable the creation of oxidative and reducing zones within the bioreactor system.

13. The circulating bioreactor system of claim 1, wherein the wireless sensors include height sensors configured to detect foam levels in the extraction vessel and transmit data to the controller for foam control.

14. The circulating bioreactor system of claim 1, wherein the wireless sensors are configured to measure optical density to monitor cell growth and adjust nutrient introduction accordingly.

15. The circulating bioreactor system of claim 1, wherein the wireless sensors are configured to monitor and optimize phase separation between the media and organic solvent in the extraction vessel.

16. The circulating bioreactor system of claim 1, wherein the wireless sensors include sensor arrays.

17. The circulating bioreactor system of claim 1, wherein the controller activates or adjusts a flow controller allowing more or less gas to the one or more synthesis vessels based on the data of the process parameters received from the wireless sensors.

18. The circulating bioreactor system of claim 1, wherein the wireless sensors include a level height sensor for detecting foam generation in the extraction vessel.

19. The circulating bioreactor system of claim 18, wherein the level height sensor is an optical line break sensor.

20. The circulating bioreactor system of claim 1, wherein the wireless sensors include sensors configured to monitor air in the one or more synthesis vessels, sensors configured to monitor waste of the bioreactor system, sensors configured to monitor product of the bioreactor system, and sensors configured to monitor a feed from a feed reservoir of the bioreactor system.

21. The circulating bioreactor system of claim 1, wherein the wireless sensors include sensors for monitoring a solvent layer and a media reservoir layer within the extraction vessel.

22. The circulating bioreactor system of claim 1, wherein the wireless sensors include sensors that are embedded in a reactor base or wall of the bioreactor system.

23. A method of operating a circulating bioreactor system for producing chemical products, the method comprising:
wireless sensors monitoring process parameters, wherein the wireless sensors are operably deployed within the bioreactor system, and the bioreactor system comprises one or more synthesis vessels configured to support the growth of microorganisms and facilitate the conversion of a carbon source into a desired product, an extraction vessel configured to extract the desired product from the media into an organic solvent, and a controller; and
the wireless sensors transmitting data of the process parameters to the controller.

24. The method of claim 23, further comprising the controller activating or adjusting a flow controller allowing more or less gas to the one or more synthesis vessels based on the data of the process parameters received from the wireless sensors.

25. The method of claim 23, further comprising detecting a level height with the wireless sensors in the extraction vessel.

26. The method of claim 23, further comprising, with the wireless sensors, monitoring air in the one or more synthesis vessels, monitoring waste of the bioreactor system, monitoring product of the bioreactor system, and/or monitoring a feed from a feed reservoir of the bioreactor system.

27. The method of claim 23, further comprising monitoring a solvent layer and/or a media reservoir layer within the extraction vessel with the wireless sensors.

28. The method of claim 23, further comprising the wireless sensors circulating within the bioreactor system and/or being are embedded in a reactor base or wall of the bioreactor system.


SEQUENCE LISTING (30 sequences)

SEQ ID NO: 1 - DNA, 3563 bp, synthetic construct
  misc_feature 1..3563: note = Description of sequence: MVA1 (mvaE:mvaS)
  source 1..3563: mol_type = other DNA
atgaagacagtcgttatcatcgacgctctacgaacccctatcgggaagtacaagggatctctatctcaggtttctgcagtcgatcttggaactcatgttactactcagctccttaagcggcattcgactatctctgaggagatcgaccaggtaatcttcggaaacgtactgcaggcagggaacggtcagaacccagctcgacaaatcgcgatcaactctggactgagtcacgagatccctgcaatgaccgtaaacgaggtctgcggttctggtatgaaggctgttatcctagctaagcagctaatccagctgggtgaagcagaggtgctaatcgcgggtggtatcgagaacatgtctcaggcgccaaagctacagagattcaactacgagactgagtcatacgacgctcctttctctagcatgatgtacgacggtctaactgacgctttcagtggacaggcaatgggattgacagcagaaaacgttgcagaaaagtaccatgttacacgggaggagcaagaccagttctcggtccactcccagctgaaggcggcgcaggcgcaagcagaggggatcttcgcggatgagatcgcacccctagaggtctcaggtactctcgttgagaaggacgaggggatcagaccaaacagtagcgtggagaagctcggaacgctaaagactgtattcaaggaggacggaactgttacagcggggaacgctagtactatcaacgacggagcgtcggcgctcatcatcgcttcgcaagagtacgcggaagcgcacggtctcccctacctggcaatcatcagagacagtgtcgaggtcggtatcgatcctgcatacatggggatcagccccatcaaggctatccaaaagttgctggcacgtaaccaactcacgacagaagagatcgatctttacgagatcaacgaggcgttcgcagctacatctatcgtagtccagcgtgagctcgctctacctgaagagaaggtaaacatctacgggggtggtatcagtttgggacacgcaatcggagcgactggggctaggctactaactagcctttcataccagctaaaccagaaggagaagaagtacggtgtagcttccctatgcatcggtggtgggctaggactcgcaatgcttcttgagcggccccaacagaagaagaactcgcgattctaccagatgagccccgaggaaaggcttgcttccttgctaaacgagggtcaaatctctgcagacactaagaaggagttcgagaacacggctttgtcctcgcagatcgcgaaccacatgatcgagaaccaaatctctgaaacagaggttccaatgggagtggggctgcatctaaccgtagacgagactgactacctagtcccaatggcgaccgaggagccctccgttatcgcagctctctctaacggggcaaagatcgcgcaggggttcaagaccgttaaccagcaaagactgatgcgtggtcaaatcgtattctacgacgtggcggacgcggaatccctcatcgacgaactacaggtcagggaaacagagatcttccagcaggcagagcttagctacccttcaatcgtaaagagaggaggtggactacgggatcttcagtaccgagctttcgacgaatcgttcgtaagtgttgacttcctagttgacgtaaaggatgcgatgggagctaacatcgttaacgcaatgctagagggtgttgcggagctcttccgagagtggttcgcggaacagaagatcctattctctatcttgagtaactacgctacggagtctgtggtaacaatgaagacggctatccccgtttcccggctttcgaagggaagtaacgggagggaaatcgcggagaagatcgtgcttgcttctagatacgcttctctggatccttacagggctgttacgcacaacaagggtatcatgaacgggatcgaggcggttgtattggcaaccgggaacgacactcgggctgtttctgctagttgtcacgcattcgcagtaaaggagggacggtaccagggattgacaagttggactctcgacggggaacagctaatcggagagatctctgtgcccctcgctctggcgacggtagggggtgcgactaaggttctgccaaagtctcaggctgcagcggaccttctagctgtaactgatgcgaaggagctttcgcgtgttgttgcagctgtgggactagctcagaaccttgctgcgctacgggcgttggtcagtgagggaatccagaagggacatatggcgctacaggcacgatcgttggctatgacggttggagctactgggaaggaagtggaggcagtcgcgcagcaactaaagagacagaagactatgaaccaggaccgtgctctagcgatcctgaacgacctgcgaaagcagtaatgacaatcggtatcgacaagatctcgttcttcgttcctccttactacatcgatatgactgctctcgcggaggcacgaaacgtagaccccggtaagttccacatcggaatcggtcaggaccagatggcggttaaccccatcagccaggacatcgttaccttcgctgcgaacgcagctgaggcgatccttactaaggaagacaaggaggcgatcgacatggttatcgttggaactgagagcagcatcgatgaatcaaaggctgcagctgtagttctacatcgacttatggggatccagccattcgctcgaagtttcgagatcaaggagggttgttacggagctacagcgggtctacagcttgctaagaaccacgtagctctacacccagataagaaggttctagtagtcgcagcggatatcgctaagtacggattgaactccgggggtgagccaactcagggagcaggggctgtggcgatgctggtggctagcgaacctagaatcctcgcactaaaggaggataacgttatgttgacgcaagatatctacgacttctggcgtccaacaggtcacccctacccaatggttgacggtccactatccaacgagacttacatccagtctttcgctcaggtatgggacgagcataagaagagaacggggttggatttcgctgactacgacgctcttgcattccacatcccatacaccaagatgggtaagaaggcgttgctggctaagatcagcgaccagacagaggcggaacaggaaagaatcttggcaagatacgaagaatcaatcatctactctagacgagtagggaacctatacactggatctctctacttgggtcttatctcgctactagagaacgctacgacgctgacagcgggaaaccaaatcgggctattcagctacgggtcgggtgcagttgcagagttcttcacaggggagctagtagctggataccaaaaccacctacagaaggaaacgcacttggctctcctagacaacagaaccgagctttccatcgcggaatacgaggctatgttcgctgagactttggacaccgacatcgatcagactcttgaggatgaattgaagtactcgatcagtgctatcaacaacacggtacgttcctaccgaaactga

SEQ ID NO: 2 - DNA, 3387 bp, synthetic construct
  misc_feature 1..3387: note = Description of sequence: MVA2 (ldi:mvaK2:mvaD:mvaK1)
  source 1..3387: mol_type = other DNA
atgcagacggagcacgtaatcctcctcaacgctcaaggtgtacctacaggaacgttggagaagtacgcggcacacactgctgacacaagactccaccttgctttctcaagttggttgttcaacgcaaagggtcagctactagtcaccagacgagctctatctaagaaggcatggcccggtgtttggactaactctgtctgcggtcacccacagctaggtgaatctaacgaggacgcagtaatcagaaggtgccgctacgagctcggagttgaaatcacacctccagaatctatctaccctgatttccgatacagggcgacagacccaagtggcatcgtagagaacgaggtgtgcccagtcttcgccgctagaacaactagcgccctccagatcaacgacgacgaggtaatggattaccagtggtgcgacctagcagacgttctacacgggatcgatgcgactccatgggccttcagtccttggatggttatgcaagctacgaaccgtgaggcacgtaagcgtctttctgcattcacccagctaaagtaatgatcgcagtaaagacatgcgggaagctttactgggcaggagagtacgcgatcctagaaccagggcagcttgctctcatcaaggacatccccatctacatgagggcggagatcgcattctcggattcttaccgaatctacagtgacatgttcgacttcgctgttgacctacgtcctaaccccgactacagtctcatccaggagacgatcgcattgatgggggatttcttggctgtaagaggacagaacctacgtccattctcactaaagatctgcggaaagatggagcgggagggaaagaagttcggtttgggctcaagcggttctgttgtggtcctagttgtcaaggcattgttggcgctctacaacctaagtgtagaccagaaccttctcttcaagttgactagcgcagtacttcttaagcggggtgacaacggctctatgggggaccttgcatgtatcgttgctgaggatctagtagtttaccaatctttcgacaggcaaaaggcagcggcatggctagaggaggagaacctagcaaccgttctagagagagactgggggttcttcatctcacaggttaagccaactctagagtgcgacttcctagttgggtggactaaggaagtcgcagtaagctcacatatggttcagcagatcaagcagaacatcaaccagaacttcttgtcgtcatccaaggagactgttgtatctttggttgaagctttggagcagggaaaggctgaaaaggttatcgagcaagtagaggtcgctagtaagctactagagggtctttcgactgacatctacacacccctactaaggcagctaaaggaggcgtctcaggaccttcaggctgttgctaagtcatctggcgctggtggcggtgactgcggtatcgctcttagcttcgacgctcagagtacgaagactttgaagaacagatgggcggatctaggaatcgagctcctctaccaggaaaggatcggtcacgatgataagtcttgatggatcgagagcctgtaactgtcagaagttacgctaacatcgcgatcatcaagtactggggtaagaagaaggagaaggaaatggttcctgcgacctcttcaatcagcctcacactagaaaacatgtacaccgagacaactctcagtcccctcccagcgaacgttactgcagatgagttctacatcaacggacagctacaaaacgaagtagaacatgcgaagatgtctaagatcatcgaccggtacagacctgcaggagaggggttcgttcggatcgacacccaaaacaacatgcctacagctgcgggtctatcgtcctcttcgtctggactaagtgctctcgtcaaggcatgcaacgcgtacttcaagctaggactcgatagaagtcaactcgcacaggaggcgaagttcgcaagtggaagctcttctcggagtttctacggaccacttggtgcttgggacaaggactctggtgagatctaccccgttgagaccgatctaaagctagctatgatcatgcttgttctagaggacaagaagaagcctatctcgtcgagagatgggatgaagctctgtgtcgaaactagcactacattcgatgactgggtaaggcagagtgaaaaggactaccaggacatgcttatctacctcaaggaaaacgacttcgcgaagatcggtgagcttacggaaaagaacgcactagcgatgcacgctacaactaagactgcatctcccgcgttcagctaccttactgacgcatcttacgaggctatggacttcgtaagacagttgcgtgagaagggggaggcgtgctacttcactatggacgctggtcccaacgttaaggtattctgccaagagaaggacctcgagcacttgtccgagatcttcggccacaggtacaggctaatcgtttccaagaccaaggacctatcgcaagatgactgctgctaatgactaagaaggtgggagttggacaagcacactcaaagatcatcctcatcggtgaacacgcggtagtatacggatacccagcaatcagtctccctcttctcgaggttgaggtaacttgtaaggttgtgcccgctgaatctccctggcgtctctacgaagaagatactctatctatggcagtatacgcgtcactcgagtacttgaacatcactgaagcttgcatcagatgcgagatcgactcggcgatccctgagaagcgaggtatgggtagttctgcggctatctcaatcgctgcaatccgtgcagtattcgattactaccaggctgaccttcctcatgatgtactagagatccttgtaaacagagcggagatgatcgcacatatgaacccctctggtcttgatgctaagacgtgcctttctgatcaaccaatccggttcatcaagaacgttggattcactgagttggaaatggatctctcggcgtacctcgtaatcgcggacactggcgtatacggtcacactcgtgaggctatccaggtcgtacagaacaaggggaaggacgcgctacccttcctacatgcattgggtgagttgactcagcaggctgaagttgcaatcagtcagaaggacgcggagggacttggtcagatccttagccaggcgcatttgcaccttaaggagatcggagtttcgtctccagaagcggatttcctagtagagactacactctctcacggtgcgcttggcgctaagatgagtggcggcggtctcggaggctgtatcatcgcgctagtcacaaacctcactcatgcacaggagctagcagagcggcttgaggagaagggtgctgtacaaacttggatcgagtctctttga

SEQ ID NO: 3 - DNA, 2911 bp, synthetic construct
  misc_feature 1..2911: note = Description of sequence: CRT1.1 (crtE:blh:crtY)
  source 1..2911: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcatgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaagggatga

SEQ ID NO: 4 - DNA, 3307 bp, synthetic construct
  misc_feature 1..3307: note = Description of sequence: CRT2.1 (crtI:crtB:ispA)
  source 1..3307: mol_type = other DNA
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaatgatgagccaacctccgttactcgaccatgcaactcagactatggccaacggatctaagtcgttcgcaacagcagctaagctcttcgacccagctacacgtcgatcggtgctaatgttgtacacctggtgcagacattgtgatgacgtaatcgacgatcagacacacggtttcgcctcagaagccgctgccgaagaggaagctacacagcgactagccaggttgcgcacattgacccttgcagctttcgagggtgctgagatgcaagacccggcattcgcggcctttcaagaggttgccctgacccacggtatcactcctcgtatggccttggatcacctagacggcttcgccatggacgttgcgcaaaccaggtatgttacttttgaagataccctaaggtactgctatcacgtcgccggcgttgtgggtcttatgatggctcgtgtaatgggtgtgcgtgatgaacgtgtactagatagggcttgtgacctaggactagcattccagctgaccaacatcgcacgagacatcatcgacgatgcagcaatcgatcgctgctacctccccgcagagtggctgcaggacgccggactgaccccggagaactatgctgccagggagaaccgtgccgcactagctcgagtagctgagcgcctaatcgacgcagcagagccatactacatctcaagccaggcagggcttcatgacctgccacctcgttgcgcttgggccatcgctacagctcgctcggtctacagggagatcgggatcaaggtaaaggccgctggaggttcggcctgggacagacgtcagcataccagcaagggtgagaagatcgccatgctaatggccgcaccgggacaagttatccgtgcaaaaactacgcgtgttactccacggccagctggtctttggcaacggcctgtctaatggacttccctcagcaacttgaggcttgcgtcaagcaggcgaaccaggctttgtctaggttcatcgcgcctctacccttccagaacacacctgtagtagagaccatgcagtacggtgcactattgggtggaaagcgactaagacctttcttggtttacgctactggtcacatgttcggagtctcaacgaacacgctggatgcacctgcagctgcagtagagtgtatccacgcatactcgctaatccacgacgatctcccggctatggacgacgacgacctacgtcgcggattgcctacttgccacgtaaagttcggcgaggcaaacgcaatcctcgcaggtgacgctctccagacactggccttcagtatcttgtcggatgcggatatgccagaggtatctgatagagatcgaatctctatgatctcggagttggcatctgcgagcggaatcgcagggatgtgcggaggacaggctcttgatcttgacgctgagggtaagcatgttcccctggacgcactcgagaggatccacagacacaagactggtgcactcatccgagctgctgtccgtctcggagcgctcagcgctggggacaagggacgaagggcactgccagtactagacaagtacgcagagtctatcggactagctttccaggtacaggatgatatcctagatgttgttggtgacactgctactctaggtaagaggcagggggcagaccagcagttggggaagagtacttaccccgcacttctagggttggagcaggcgaggaagaaggcgagagacctgatcgacgatgctcgccagagtctaaagcaattggctgagcagtcgttggatacatctgctcttgaggcccttgccgactacatcatccaacgaaacaagtga

SEQ ID NO: 5 - DNA, 2412 bp, synthetic construct
  misc_feature 1..2412: note = Description of sequence: mvaE
  source 1..2412: mol_type = other DNA
atgaagacagtcgttatcatcgacgctctacgaacccctatcgggaagtacaagggatctctatctcaggtttctgcagtcgatcttggaactcatgttactactcagctccttaagcggcattcgactatctctgaggagatcgaccaggtaatcttcggaaacgtactgcaggcagggaacggtcagaacccagctcgacaaatcgcgatcaactctggactgagtcacgagatccctgcaatgaccgtaaacgaggtctgcggttctggtatgaaggctgttatcctagctaagcagctaatccagctgggtgaagcagaggtgctaatcgcgggtggtatcgagaacatgtctcaggcgccaaagctacagagattcaactacgagactgagtcatacgacgctcctttctctagcatgatgtacgacggtctaactgacgctttcagtggacaggcaatgggattgacagcagaaaacgttgcagaaaagtaccatgttacacgggaggagcaagaccagttctcggtccactcccagctgaaggcggcgcaggcgcaagcagaggggatcttcgcggatgagatcgcacccctagaggtctcaggtactctcgttgagaaggacgaggggatcagaccaaacagtagcgtggagaagctcggaacgctaaagactgtattcaaggaggacggaactgttacagcggggaacgctagtactatcaacgacggagcgtcggcgctcatcatcgcttcgcaagagtacgcggaagcgcacggtctcccctacctggcaatcatcagagacagtgtcgaggtcggtatcgatcctgcatacatggggatcagccccatcaaggctatccaaaagttgctggcacgtaaccaactcacgacagaagagatcgatctttacgagatcaacgaggcgttcgcagctacatctatcgtagtccagcgtgagctcgctctacctgaagagaaggtaaacatctacgggggtggtatcagtttgggacacgcaatcggagcgactggggctaggctactaactagcctttcataccagctaaaccagaaggagaagaagtacggtgtagcttccctatgcatcggtggtgggctaggactcgcaatgcttcttgagcggccccaacagaagaagaactcgcgattctaccagatgagccccgaggaaaggcttgcttccttgctaaacgagggtcaaatctctgcagacactaagaaggagttcgagaacacggctttgtcctcgcagatcgcgaaccacatgatcgagaaccaaatctctgaaacagaggttccaatgggagtggggctgcatctaaccgtagacgagactgactacctagtcccaatggcgaccgaggagccctccgttatcgcagctctctctaacggggcaaagatcgcgcaggggttcaagaccgttaaccagcaaagactgatgcgtggtcaaatcgtattctacgacgtggcggacgcggaatccctcatcgacgaactacaggtcagggaaacagagatcttccagcaggcagagcttagctacccttcaatcgtaaagagaggaggtggactacgggatcttcagtaccgagctttcgacgaatcgttcgtaagtgttgacttcctagttgacgtaaaggatgcgatgggagctaacatcgttaacgcaatgctagagggtgttgcggagctcttccgagagtggttcgcggaacagaagatcctattctctatcttgagtaactacgctacggagtctgtggtaacaatgaagacggctatccccgtttcccggctttcgaagggaagtaacgggagggaaatcgcggagaagatcgtgcttgcttctagatacgcttctctggatccttacagggctgttacgcacaacaagggtatcatgaacgggatcgaggcggttgtattggcaaccgggaacgacactcgggctgtttctgctagttgtcacgcattcgcagtaaaggagggacggtaccagggattgacaagttggactctcgacggggaacagctaatcggagagatctctgtgcccctcgctctggcgacggtagggggtgcgactaaggttctgccaaagtctcaggctgcagcggaccttctagctgtaactgatgcgaaggagctttcgcgtgttgttgcagctgtgggactagctcagaaccttgctgcgctacgggcgttggtcagtgagggaatccagaagggacatatggcgctacaggcacgatcgttggctatgacggttggagctactgggaaggaagtggaggcagtcgcgcagcaactaaagagacagaagactatgaaccaggaccgtgctctagcgatcctgaacgacctgcgaaagcagtga

SEQ ID NO: 6 - DNA, 1152 bp, synthetic construct
  misc_feature 1..1152: note = Description of sequence: mvaS
  source 1..1152: mol_type = other DNA
atgacaatcggtatcgacaagatctcgttcttcgttcctccttactacatcgatatgactgctctcgcggaggcacgaaacgtagaccccggtaagttccacatcggaatcggtcaggaccagatggcggttaaccccatcagccaggacatcgttaccttcgctgcgaacgcagctgaggcgatccttactaaggaagacaaggaggcgatcgacatggttatcgttggaactgagagcagcatcgatgaatcaaaggctgcagctgtagttctacatcgacttatggggatccagccattcgctcgaagtttcgagatcaaggagggttgttacggagctacagcgggtctacagcttgctaagaaccacgtagctctacacccagataagaaggttctagtagtcgcagcggatatcgctaagtacggattgaactccgggggtgagccaactcagggagcaggggctgtggcgatgctggtggctagcgaacctagaatcctcgcactaaaggaggataacgttatgttgacgcaagatatctacgacttctggcgtccaacaggtcacccctacccaatggttgacggtccactatccaacgagacttacatccagtctttcgctcaggtatgggacgagcataagaagagaacggggttggatttcgctgactacgacgctcttgcattccacatcccatacaccaagatgggtaagaaggcgttgctggctaagatcagcgaccagacagaggcggaacaggaaagaatcttggcaagatacgaagaatcaatcatctactctagacgagtagggaacctatacactggatctctctacttgggtcttatctcgctactagagaacgctacgacgctgacagcgggaaaccaaatcgggctattcagctacgggtcgggtgcagttgcagagttcttcacaggggagctagtagctggataccaaaaccacctacagaaggaaacgcacttggctctcctagacaacagaaccgagctttccatcgcggaatacgaggctatgttcgctgagactttggacaccgacatcgatcagactcttgaggatgaattgaagtactcgatcagtgctatcaacaacacggtacgttcctaccgaaactga

SEQ ID NO: 7 - DNA, 879 bp, synthetic construct
  misc_feature 1..879: note = Description of sequence: mvaK1
  source 1..879: mol_type = other DNA
atgactaagaaggtgggagttggacaagcacactcaaagatcatcctcatcggtgaacacgcggtagtatacggatacccagcaatcagtctccctcttctcgaggttgaggtaacttgtaaggttgtgcccgctgaatctccctggcgtctctacgaagaagatactctatctatggcagtatacgcgtcactcgagtacttgaacatcactgaagcttgcatcagatgcgagatcgactcggcgatccctgagaagcgaggtatgggtagttctgcggctatctcaatcgctgcaatccgtgcagtattcgattactaccaggctgaccttcctcatgatgtactagagatccttgtaaacagagcggagatgatcgcacatatgaacccctctggtcttgatgctaagacgtgcctttctgatcaaccaatccggttcatcaagaacgttggattcactgagttggaaatggatctctcggcgtacctcgtaatcgcggacactggcgtatacggtcacactcgtgaggctatccaggtcgtacagaacaaggggaaggacgcgctacccttcctacatgcattgggtgagttgactcagcaggctgaagttgcaatcagtcagaaggacgcggagggacttggtcagatccttagccaggcgcatttgcaccttaaggagatcggagtttcgtctccagaagcggatttcctagtagagactacactctctcacggtgcgcttggcgctaagatgagtggcggcggtctcggaggctgtatcatcgcgctagtcacaaacctcactcatgcacaggagctagcagagcggcttgaggagaagggtgctgtacaaacttggatcgagtctctttga

SEQ ID NO: 8 - DNA, 1008 bp, synthetic construct
  misc_feature 1..1008: note = Description of sequence: mvAK2
  source 1..1008: mol_type = other DNA
atgatcgcagtaaagacatgcgggaagctttactgggcaggagagtacgcgatcctagaaccagggcagcttgctctcatcaaggacatccccatctacatgagggcggagatcgcattctcggattcttaccgaatctacagtgacatgttcgacttcgctgttgacctacgtcctaaccccgactacagtctcatccaggagacgatcgcattgatgggggatttcttggctgtaagaggacagaacctacgtccattctcactaaagatctgcggaaagatggagcgggagggaaagaagttcggtttgggctcaagcggttctgttgtggtcctagttgtcaaggcattgttggcgctctacaacctaagtgtagaccagaaccttctcttcaagttgactagcgcagtacttcttaagcggggtgacaacggctctatgggggaccttgcatgtatcgttgctgaggatctagtagtttaccaatctttcgacaggcaaaaggcagcggcatggctagaggaggagaacctagcaaccgttctagagagagactgggggttcttcatctcacaggttaagccaactctagagtgcgacttcctagttgggtggactaaggaagtcgcagtaagctcacatatggttcagcagatcaagcagaacatcaaccagaacttcttgtcgtcatccaaggagactgttgtatctttggttgaagctttggagcagggaaaggctgaaaaggttatcgagcaagtagaggtcgctagtaagctactagagggtctttcgactgacatctacacacccctactaaggcagctaaaggaggcgtctcaggaccttcaggctgttgctaagtcatctggcgctggtggcggtgactgcggtatcgctcttagcttcgacgctcagagtacgaagactttgaagaacagatgggcggatctaggaatcgagctcctctaccaggaaaggatcggtcacgatgataagtcttga

SEQ ID NO: 9 - DNA, 954 bp, synthetic construct
  misc_feature 1..954: note = Description of sequence: mvaD
  source 1..954: mol_type = other DNA
atggatcgagagcctgtaactgtcagaagttacgctaacatcgcgatcatcaagtactggggtaagaagaaggagaaggaaatggttcctgcgacctcttcaatcagcctcacactagaaaacatgtacaccgagacaactctcagtcccctcccagcgaacgttactgcagatgagttctacatcaacggacagctacaaaacgaagtagaacatgcgaagatgtctaagatcatcgaccggtacagacctgcaggagaggggttcgttcggatcgacacccaaaacaacatgcctacagctgcgggtctatcgtcctcttcgtctggactaagtgctctcgtcaaggcatgcaacgcgtacttcaagctaggactcgatagaagtcaactcgcacaggaggcgaagttcgcaagtggaagctcttctcggagtttctacggaccacttggtgcttgggacaaggactctggtgagatctaccccgttgagaccgatctaaagctagctatgatcatgcttgttctagaggacaagaagaagcctatctcgtcgagagatgggatgaagctctgtgtcgaaactagcactacattcgatgactgggtaaggcagagtgaaaaggactaccaggacatgcttatctacctcaaggaaaacgacttcgcgaagatcggtgagcttacggaaaagaacgcactagcgatgcacgctacaactaagactgcatctcccgcgttcagctaccttactgacgcatcttacgaggctatggacttcgtaagacagttgcgtgagaagggggaggcgtgctacttcactatggacgctggtcccaacgttaaggtattctgccaagagaaggacctcgagcacttgtccgagatcttcggccacaggtacaggctaatcgtttccaagaccaaggacctatcgcaagatgactgctgctga

SEQ ID NO: 10 - DNA, 549 bp, synthetic construct
  misc_feature 1..549: note = Description of sequence: idi
  source 1..549: mol_type = other DNA
atgcagacggagcacgtaatcctcctcaacgctcaaggtgtacctacaggaacgttggagaagtacgcggcacacactgctgacacaagactccaccttgctttctcaagttggttgttcaacgcaaagggtcagctactagtcaccagacgagctctatctaagaaggcatggcccggtgtttggactaactctgtctgcggtcacccacagctaggtgaatctaacgaggacgcagtaatcagaaggtgccgctacgagctcggagttgaaatcacacctccagaatctatctaccctgatttccgatacagggcgacagacccaagtggcatcgtagagaacgaggtgtgcccagtcttcgccgctagaacaactagcgccctccagatcaacgacgacgaggtaatggattaccagtggtgcgacctagcagacgttctacacgggatcgatgcgactccatgggccttcagtccttggatggttatgcaagctacgaaccgtgaggcacgtaagcgtctttctgcattcacccagctaaagtga

SEQ ID NO: 11 - DNA, 924 bp, synthetic construct
  misc_feature 1..924: note = Description of sequence: crtE
  source 1..924: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcatga

SEQ ID NO: 12 - DNA, 930 bp, synthetic construct
  misc_feature 1..930: note = Description of sequence: crtB
  source 1..930: mol_type = other DNA
atgagccaacctccgttactcgaccatgcaactcagactatggccaacggatctaagtcgttcgcaacagcagctaagctcttcgacccagctacacgtcgatcggtgctaatgttgtacacctggtgcagacattgtgatgacgtaatcgacgatcagacacacggtttcgcctcagaagccgctgccgaagaggaagctacacagcgactagccaggttgcgcacattgacccttgcagctttcgagggtgctgagatgcaagacccggcattcgcggcctttcaagaggttgccctgacccacggtatcactcctcgtatggccttggatcacctagacggcttcgccatggacgttgcgcaaaccaggtatgttacttttgaagataccctaaggtactgctatcacgtcgccggcgttgtgggtcttatgatggctcgtgtaatgggtgtgcgtgatgaacgtgtactagatagggcttgtgacctaggactagcattccagctgaccaacatcgcacgagacatcatcgacgatgcagcaatcgatcgctgctacctccccgcagagtggctgcaggacgccggactgaccccggagaactatgctgccagggagaaccgtgccgcactagctcgagtagctgagcgcctaatcgacgcagcagagccatactacatctcaagccaggcagggcttcatgacctgccacctcgttgcgcttgggccatcgctacagctcgctcggtctacagggagatcgggatcaaggtaaaggccgctggaggttcggcctgggacagacgtcagcataccagcaagggtgagaagatcgccatgctaatggccgcaccgggacaagttatccgtgcaaaaactacgcgtgttactccacggccagctggtctttggcaacggcctgtctga

SEQ ID NO: 13 - DNA, 1479 bp, synthetic construct
  misc_feature 1..1479: note = Description of sequence: crtI
  source 1..1479: mol_type = other DNA
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaatga

SEQ ID NO: 14 - DNA, 1161 bp, synthetic construct
  misc_feature 1..1161: note = Description of sequence: crtY
  source 1..1161: mol_type = other DNA
atgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaagggatga

SEQ ID NO: 15 - DNA, 900 bp, synthetic construct
  misc_feature 1..900: note = Description of sequence: ispA
  source 1..900: mol_type = other DNA
atggacttccctcagcaacttgaggcttgcgtcaagcaggcgaaccaggctttgtctaggttcatcgcgcctctacccttccagaacacacctgtagtagagaccatgcagtacggtgcactattgggtggaaagcgactaagacctttcttggtttacgctactggtcacatgttcggagtctcaacgaacacgctggatgcacctgcagctgcagtagagtgtatccacgcatactcgctaatccacgacgatctcccggctatggacgacgacgacctacgtcgcggattgcctacttgccacgtaaagttcggcgaggcaaacgcaatcctcgcaggtgacgctctccagacactggccttcagtatcttgtcggatgcggatatgccagaggtatctgatagagatcgaatctctatgatctcggagttggcatctgcgagcggaatcgcagggatgtgcggaggacaggctcttgatcttgacgctgagggtaagcatgttcccctggacgcactcgagaggatccacagacacaagactggtgcactcatccgagctgctgtccgtctcggagcgctcagcgctggggacaagggacgaagggcactgccagtactagacaagtacgcagagtctatcggactagctttccaggtacaggatgatatcctagatgttgttggtgacactgctactctaggtaagaggcagggggcagaccagcagttggggaagagtacttaccccgcacttctagggttggagcaggcgaggaagaaggcgagagacctgatcgacgatgctcgccagagtctaaagcaattggctgagcagtcgttggatacatctgctcttgaggcccttgccgactacatcatccaacgaaacaagtga

SEQ ID NO: 16 - DNA, 828 bp, synthetic construct
  misc_feature 1..828: note = Description of sequence: blh
  source 1..828: mol_type = other DNA
atgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactga

SEQ ID NO: 17 - DNA, 951 bp, synthetic construct
  misc_feature 1..951: note = Description of sequence: RDH12
  source 1..951: mol_type = other DNA
atgctggtcacactagggctactaacatcattcttctcgttcctgtacatggttgcgccttctattaggaagttctttgcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagtga

SEQ ID NO: 18 - DNA, 862 bp, synthetic construct
  misc_feature 1..862: note = Description of sequence: RDH12-shortGCAGGAGGAGT
  source 1..862: mol_type = other DNA
atgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagtga

SEQ ID NO: 19 - DNA, 810 bp, synthetic construct
  misc_feature 1..810: note = Description of sequence: yBBO
  source 1..810: mol_type = other DNA
atgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggatga

SEQ ID NO: 20 - DNA, 912 bp, synthetic construct
  misc_feature 1..912: note = Description of sequence: rdh12-His6
  source 1..912: mol_type = other DNA
atggcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagcttaccgccgcactcgagcatcaccatcaccatcactga

SEQ ID NO: 21 - DNA, 846 bp, synthetic construct
  misc_feature 1..846: note = Description of sequence: ybbO-His6
  source 1..846: mol_type = other DNA
atgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggacttaccgccgcactcgagcatcaccatcaccatcactga

SEQ ID NO: 22 - DNA, 3786 bp, synthetic construct
  misc_feature 1..3786: note = Description of sequence: CRT1.2 (crtE:blh:crtY:rdh12)
  source 1..3786: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatggcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagtga

SEQ ID NO: 23 - DNA, 3822 bp, synthetic construct
  misc_feature 1..3822: note = Description of sequence: CRT1.3 (crtE:blh:crtY:rdh12-his6)
  source 1..3822: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatggcaggaggagtatgccgaactaacgtgcagctacctggaaaggtcgtggttatcactggggcgaacactgggatcggtaaggagacggcacgtgagctcgcgtcgcgtggggctcgtgtatacatcgcatgcagggacgtcctgaagggtgagtctgctgcatccgagatccgagtagacaccaagaactcgcaggtgctagtacgaaagctcgacttgagcgacactaagagtatcagggcgttcgctgaaggtttcctggcagaggagaagcaacttcacatcctgatcaataacgcaggtgttatgatgtgcccgtactcgaaaacagcggacggattcgagacccatctaggtgtaaaccaccttggacacttcttgctgacttacttgctactagagcgactcaaggtttctgcgccagcaagggtcgtcaacgtatcgtcggtagcgcaccatatcgggaagatcccatttcacgacctacagtcggagaagagatactcgcgaggtttcgcttactgccactcgaagctagctaacgttctcttcactcgggaattggctaagcgtttgcaagggacaggtgtaacgacatacgcggttcaccctggggttgtcaggtctgagcttgttagacattcgtctcttctttgtctactatggcgtctattcagtcctttcgtgaagacagctcgggagggggctcagactagcttgcactgtgctctggcggaagggctagaacctctatctggaaagtacttcagtgattgcaagcggacctgggtgagtccaagagcgagaaacaacaagactgctgagagattgtggaatgtcagctgcgagctacttggaatccgatgggagcttaccgccgcactcgagcatcaccatcaccatcactga

SEQ ID NO: 24 - DNA, 3720 bp, synthetic construct
  misc_feature 1..3720: note = Description of sequence: CRT1.4 (crtE:blh:crtY:ybbO)
  source 1..3720: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggatga

SEQ ID NO: 25 - DNA, 3756 bp, synthetic construct
  misc_feature 1..3756: note = Description of sequence: CRT1.5 (crtE:blh:crtY:ybbO-his6)
  source 1..3756: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttgatgggacttatgcttatcgattggtgcgcattggcccttgttgtattcatcggtctacctcacggagcacttgatgcggctatctcattctctatgatctcgtcggcaaagcgaatcgctcgtctagccggaatcctattgatctacctcctactagccactgcattcttccttatctggtaccagctgcctgctttctcgctactaatcttcctcctcatctcaatcatccacttcggcatggcagacttcaacgccagccctagtaagctaaagtggccccacatcatcgctcacggtggggtggtgacggtgtggcttccccttatccaaaagaacgaagtaacgaagcttttctctatccttactaacggtccgactccaatcctttgggacatcctattgatcttcttcttgtgctggtctatcggcgtttgtcttcacacttacgagacattgcgtagcaagcactacaacatcgcattcgagctaatcggtttgatcttcctagcgtggtacgctcctcctctcgttacattcgctacctacttctgcttcatccactctaggagacatttcagtttcgtatggaagcaattgcaacacatgtcaagtaagaagatgatgatcggaagtgcaatcatcctatcttgcacgtcttggctcatcggaggcggaatctacttcttcctaaacagcaagatgatcgctagtgaggctgctctacaaactgtcttcatcggactcgccgctctgaccgtgcctcatatgatcttgatcgatttcatcttccgtcctcattcttcgcggatcaagatcaagaactaatgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtgatgactcacaaggctacagagatcctaacagggaaggttatgcaaaagagtgtcctaatcactggttgcagttctggtatcggcctagagtcggctctagaactcaagagacagggtttccacgtgctggcgggatgcaggaagccagatgacgtcgaacgcatgaacagcatgggtttcactggagtattgatcgaccttgacagccccgagagtgtcgacagagcagcagatgaggttatcgcacttacagacaactgtctatacggtatcttcaacaacgcaggtttcggaatgtacggacctctatcgactatcagtcgagcacagatggaacagcagttcagtgcgaacttcttcggcgcgcaccagttgacaatgagacttcttcccgctatgttgccgcacggggaaggtcgcatcgtaatgacgtcttctgtaatgggactaatctcgacgcctggtagaggtgcgtacgcagcttcgaagtacgccttggaggcatggagcgacgcgctgcgtatggaacttaggcactcggggatcaaggttagcctgatcgagcctgggccaatccgcactcgattcaccgacaacgttaaccaaactcaatcagacaagcccgtcgaaaaccccggtatcgcggcacgattcacgcttggacctgaggccgtagttgacaaggttcgtcacgcattcatcagtgagaagccgaagatgcgatacccagtaactctggtaacctgggctgttatggttctaaagcgcctgctgccgggaagggtaatggataagatcctacagggacttaccgccgcactcgagcatcaccatcaccatcactga

SEQ ID NO: 26 - DNA, 3307 bp, synthetic construct
  misc_feature 1..3307: note = Description of sequence: CRT2.2 (crtI:crtB:ispA)
  source 1..3307: mol_type = other DNA
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaataatgagccaacctccgttactcgaccatgcaactcagactatggccaacggatctaagtcgttcgcaacagcagctaagctcttcgacccagctacacgtcgatcggtgctaatgttgtacacctggtgcagacattgtgatgacgtaatcgacgatcagacacacggtttcgcctcagaagccgctgccgaagaggaagctacacagcgactagccaggttgcgcacattgacccttgcagctttcgagggtgctgagatgcaagacccggcattcgcggcctttcaagaggttgccctgacccacggtatcactcctcgtatggccttggatcacctagacggcttcgccatggacgttgcgcaaaccaggtatgttacttttgaagataccctaaggtactgctatcacgtcgccggcgttgtgggtcttatgatggctcgtgtaatgggtgtgcgtgatgaacgtgtactagatagggcttgtgacctaggactagcattccagctgaccaacatcgcacgagacatcatcgacgatgcagcaatcgatcgctgctacctccccgcagagtggctgcaggacgccggactgaccccggagaactatgctgccagggagaaccgtgccgcactagctcgagtagctgagcgcctaatcgacgcagcagagccatactacatctcaagccaggcagggcttcatgacctgccacctcgttgcgcttgggccatcgctacagctcgctcggtctacagggagatcgggatcaaggtaaaggccgctggaggttcggcctgggacagacgtcagcataccagcaagggtgagaagatcgccatgctaatggccgcaccgggacaagttatccgtgcaaaaactacgcgtgttactccacggccagctggtctttggcaacggcctgtctaatggacttccctcagcaacttgaggcttgcgtcaagcaggcgaaccaggctttgtctaggttcatcgcgcctctacccttccagaacacacctgtagtagagaccatgcagtacggtgcactattgggtggaaagcgactaagacctttcttggtttacgctactggtcacatgttcggagtctcaacgaacacgctggatgcacctgcagctgcagtagagtgtatccacgcatactcgctaatccacgacgatctcccggctatggacgacgacgacctacgtcgcggattgcctacttgccacgtaaagttcggcgaggcaaacgcaatcctcgcaggtgacgctctccagacactggccttcagtatcttgtcggatgcggatatgccagaggtatctgatagagatcgaatctctatgatctcggagttggcatctgcgagcggaatcgcagggatgtgcggaggacaggctcttgatcttgacgctgagggtaagcatgttcccctggacgcactcgagaggatccacagacacaagactggtgcactcatccgagctgctgtccgtctcggagcgctcagcgctggggacaagggacgaagggcactgccagtactagacaagtacgcagagtctatcggactagctttccaggtacaggatgatatcctagatgttgttggtgacactgctactctaggtaagaggcagggggcagaccagcagttggggaagagtacttaccccgcacttctagggttggagcaggcgaggaagaaggcgagagacctgatcgacgatgctcgccagagtctaaagcaattggctgagcagtcgttggatacatctgctcttgaggcccttgccgactacatcatccaacgaaacaagtga

SEQ ID NO: 27 - DNA, 924 bp, synthetic construct
  misc_feature 1..924: note = Description of sequence: crtE*
  source 1..924: mol_type = other DNA
atggttagtggttcaaaggctggcgtatctccacatagggaaatcgaggtcatgcgacaatcaatcgacgatcacctagcaggcctgttgcccgagactgacagccaggatatcgtctcgttggccatgcgtgagggagttatggcgcctgggaagagaattcggcctttactgatgttgctagctgcacgcgacctcagataccaaggtagtatgcccacattgctcgatctagcctgcgcagtcgagctgacacatactgctagcctaatgctagatgacatgccatgtatggataacgcagagctacgtcgcgggcaacccacaacccacaagaaattcggtgagtctgtagctatcctagcaagcgtcggtctgcttagtaaggcattcggtttaatcgctgccactggtgacctgccaggagagagacgggctcaagccgtaaacgagctaagtactgcagtaggagttcagggtctagtactagggcaattccgagacctaaacgacgctgccctcgatagaacaccagacgcaatcttgagcaccaaccacctaaagacaggcatccttttcagtgctatgctacaaatcgtcgcaatcgcttcagcctcgtctccgtctactcgagagactcttcatgctttcgctctagacttcggccaggctttccaactacttgacgaccttcgagacgatcacccggagacaggtaaagaccgcaacaaagacgctggaaagtccactctagtaaacagacttggtgcggacgctgctagacagaagcttcgagagcatatcgacagtgcagataagcatctcactttcgcatgtccacaaggcggtgccatcagacagttcatgcacttgtggttcggtcaccacttggctgactggtctccggtaatgaagatcgcttga

SEQ ID NO: 28 - DNA, 1161 bp, synthetic construct
  misc_feature 1..1161: note = Description of sequence: crtY*
  source 1..1161: mol_type = other DNA
atgagagacttgatcctcgtcggcggcggtctagccaacggtcttatcgcctggcgtttgaggcagcgctacccacagctaaacttgcttctgatcgaagctggagaacagcctggaggtaaccatacttggagtttccacgaggacgatttgacacctggacagcatgcttggttggcacctctagtggctcacgcatggccgggttatgaggtgcagttcccagacctccgacgacgtctggcaagaggatactactctatcacttcggagcgcttcgccgaagcactccaccaagcacttggagaaaacatctggctaaactgctcagtgtcagaggttctaccaaatagtgtaaggctcgctaacggggaagctctattagccggggccgttatcgacggtaggggtgtaacggcctcttccgccatgcaaactggttaccagcttttcctaggtcagcaatggaggttgacacagcctcacggccttactgttccgatcctaatggacgcaacggtagcacagcaacagggttacaggtttgtttatacactgccactatcggccgatacgctgctaatcgaggacactaggtacgccaacgttccacaacgtgacgataacgcattgcgtcagacggtgacggactacgcgcactcgaagggatggcagcttgctcagttggagagagaagagacaggctgtctgcctatcacacttgcaggggacatccaggcactatgggcagacgctccaggagtaccgagatccggaatgagggcgggtttgttccatccaactacaggttactcacttccactagccgtggctctagccgacgccatcgctgactcgccaaggctgggaagcgtcccactttaccagctcactaggcaatttgctgagcgccactggcgccgacaaggtttctttcgcctattgaaccgtatgcttttcctagctggacgagaggagaatcgctggcgtgtaatgcagcgtttctacggtcttccagagccaacggtcgagaggttctacgcaggaagacttagtctattcgataaggctaggatccttacaggcaagcctccggtaccactaggagaagcttggagagccgcactaaaccattttcctgacagaagagacaaggggtga

SEQ ID NO: 29 - DNA, 1479 bp, synthetic construct
  misc_feature 1..1479: note = Description of sequence: crtI*
  source 1..1479: mol_type = other DNA
atgaagaaaacagttgtcatcggcgcaggcttcgggggacttgcattggctatcagactgcaggccgcgggaatccccacagtcctattggagcagagagacaagccaggagggcgcgcatacgtctggcatgaccagggattcacttttgatgcaggtccgacggtaatcacagatcctactgcccttgaggctcttttcacactagctggtagaagaatggaggactacgtacgtctgttgcctgttaaacccttctaccgtttgtgttgggaaagcggtaagacacttgactacgcaaacgactcggctgagctcgaggctcagatcacccagttcaacccaagagatgtagaaggatacagaaggttccttgcgtactctcaggctgtttttcaggaaggataccttcgactaggatctgttccgtttctatcttttcgagacatgctacgcgctgggccacagttgctaaagctacaggcttggcagtcggtctaccagtcagtttcgaggttcatcgaggatgaacacctcagacaggctttctctttccactcgttactggttggtggtaacccattcactacaagttcgatttacaccctaatccacgccctagagagagagtggggggtttggttccctgagggcggcactggtgccttggtgaacggcatggtcaagctattcacggaccttggcggggaaattgaacttaacgccagagtcgaagaactagtggtagcagataatcgagttagccaagtcaggctggccgacggacgcatcttcgatactgacgctgtcgcttcgaacgcagacgtagtgaacacttacaagaagcttctcggccatcatccggtcggacagaaaagagctgcggcactggaacgtaagtcaatgtcgaactctctattcgtgttgtacttcggtctgaaccaaccacactctcagttggcccaccataccatctgttttggtccacgctaccgagagctaatcgatgagatcttcacaggaagcgctcttgcagacgacttttccctatacttgcattccccatgcgttactgacccatccctagctccaccaggatgtgctagtttctacgtgctagctcctgtgccacacctgggaaacgctccgctggattgggctcaggaaggaccaaagctgcgcgacaggattttcgattacctagaagaacgttacatgccaggcttgcgatctcagttggtaacacagcgtatcttcaccccagcagacttccacgatacgctagacgctcacctagggagtgctttcagcatcgaaccgctgttgacacaaagcgcctggttccgaccacacaaccgagactctgacatcgctaacctgtacctcgtgggtgcgggaactcaccctggggccggcatccctggtgtagtcgcttctgccaaggcaacagcatcattgatgatcgaagacctgcaataa

SEQ ID NO: 30 - protein, 291 aa, synthetic construct
  REGION 1..291: note = Description of sequence: RDH12-short
MAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFSDCKRTWVSPRARNNKTAERLWNVSCELLGIRWE